(19) United States
(12) Patent Application Publication
(10) Pub. No.: US 2023/0190680 A1
(43) Pub. Date: Jun. 22, 2023

(54) METHODS AND COMPOSITIONS FOR TREATING AN RNA VIRUS INDUCED DISEASE

(71) Applicant: Golden Biotechnology Corporation, Jersey City, NJ (US)

(72) Inventors: Sheng-Yung Liu, New Taipei City (TW); Ching-Tien Su, New Taipei City (TW); Wu-Che Wen, New Taipei City (TW); Pei-Ni Chen, New Taipei City (TW)

(21) Appl. No.: 17/922,967

(22) PCT Filed: May 7, 2021
(86) PCT No.: PCT/US2021/031202

§ 371 Date: Nov. 2, 2022

Related U.S. Application Data
(60) Provisional application No. 63/022,375, filed on May 8, 2020.

(51) Int. Cl. A61K 31/122; A61P 31/14

(52) U.S. Cl. CPC A61K 31/122; A61P 31/14

(57) ABSTRACT

The present invention provides methods and compositions for treating or reducing the symptoms of or preventing an RNA virus induced disease in a subject by cyclohexenone compounds.

### FIELD OF THE INVENTION

The present invention relates to a method for treating or reducing symptoms of or preventing a RNA virus induced disease, and more particularly, to a method of administering a cyclohexenone compound.

### BACKGROUND OF THE INVENTION

An RNA virus is a virus that has RNA (ribonucleic acid) as its genetic material. This nucleic acid is usually single-stranded RNA (ssRNA) but may be double-stranded RNA (dsRNA). Notable human diseases caused by RNA viruses include the common cold, influenza, SARS, MERS, COVID-19, Dengue Virus, hepatitis C, hepatitis E, West Nile fever, Ebola virus disease, rabies, polio, mumps, and measles.

An RNA virus induced disease such as an RNA viral pneumonia is a common cause accounting for many deaths. There are roughly 450 million cases of pneumonia every year. Of those case, viral pneumonia counts for about 200 million cases which includes about 100 million children and 100 million adults. Viral pneumonia is a pneumonia caused by a virus. Pneumonia is an infection that causes inflammation in one or both of the lungs. The pulmonary alveoli fill with fluid or pus making it difficult to breathe.

Coronaviruses are a group of related RNA viruses that cause diseases in mammals and birds. In humans, these viruses cause respiratory tract infections that can range from mild to lethal. Mild illnesses include some cases of the common cold (which is caused also by certain other viruses, predominantly rhinoviruses), while more lethal varieties can cause SARS, MERS, and COVID-19.

### SUMMARY OF THE INVENTION

In one aspect provided herein are methods for treating or reducing the symptoms and/or preventing a RNA virus induced disease (such as an RNA virus induced pneumonia) in a subject comprising administering to said subject a therapeutically effective amount of a cyclohexenone compound having the structure:

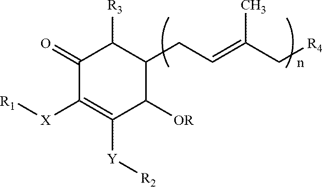

wherein each of X and Y independently is oxygen, NR₅ or sulfur;
R is a hydrogen or C(=O)C₁-C₈alkyl;
each of R₁, R₂ and R₃ independently is a hydrogen, optionally substituted methyl or
(CH₂)ₘ—CH₃;
R₄ is NR₅R₆, OR₅, OC(═O)R₇, C(═O)OR₅, C(═O)R₅, C(═O)NR₅R₆, halogen, 5 or 6-membered lactone, C₁₋C₈alkyl, C₂-C₈alkenyl, C₂-C₈alkynyl, aryl, glucosyl, wherein the 5 or 6-membered lactone, C₁₋C₈alkyl, C₂-C₈alkenyl, C₂-C₈alkynyl, aryl, and glucosyl are optionally substituted with one or more substituents selected from
NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁₋C₈alkyl, OR₅ or NR₅R₆;
m = 1-12; and
n=1-12; or a pharmaceutically acceptable salt, metabolite, solvate or prodrug thereof.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIGS. 1A/B show the study results of the expression levels reduction of HBeAg (1A) and HBsAg (1B) by an exemplary Compound 1.

FIGS. 2A/B show study results of reducing HBV NDA expression levels (2A) and HCV RNA activity (2B) by an exemplary Compound 1.

FIG. 3 illustrates the potential clinical progression of SARS-CoV-2.

FIG. 4 illustrates the multiple approaches of anti-virus, anti-inflammation and anti-fibrosis by exemplary Compound 1.

FIG. 5 provides Nrf-2 nuclear translocation study results with exemplary Compound 1 in comparison with Silymarin.

FIG. 6 provides study results of oxidative stress with exemplary Compound 1.

FIG. 7 provides study results of renal inflammation with NF-kB activation model with exemplary Compound 1.

FIG. 8 provides study results of local renal inflammation with the MCP-1, IL-6 and CD3 markers with exemplary Compound 1.

FIGS. 9A/B provide study results of anti-fibrosis activity through TGF-β1 inhibition (9A) and fibrosis-related proteins (9B) with exemplary Compound 1.

FIG. 10 provides study results of SARS inhibition by exemplary Compound 1.

FIG. 11 provides cell culture study results with exemplary Compound 1 in comparison with the control group (DMSO only).

FIGS. 12A-C provides the gene expression levels of CXCL10 (12A), IL6 (12B), and IL18 (12C), respectively.

FIGS. 13A-B provides the gene expression levels of TGFB1 (13A), and COL4A1 (13B), respectively.

### DETAILED DESCRIPTION OF THE INVENTION

Although there are many therapeutic agents developed to treat coronavirus-induced diseases (such as SARS and MERS), there is no significant effect found from the drugs developed so far.

The cyclohexenone compounds, in some embodiments, are obtained from extracts of natural products or prepared synthetically or semi-synthetically. In some embodiments, this invention provides the therapeutic and prophylactic potential of exemplary cyclohexenone compounds (e.g., Compound 1) for treating or reducing the symptoms of or preventing an RNA virus induced disease in a subject.

In some embodiments, there are provided methods for treating or reducing symptoms of and/or preventing an RNA virus induced disease (such as an RNA virus induced pneumonia) in a subject comprising administering to said subject a therapeutically effective amount of a cyclohexenone compound having the structure:

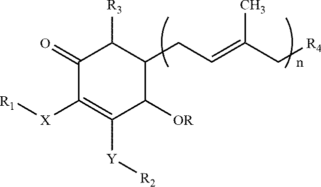

wherein each of X and Y independently is oxygen, NR₅ or sulfur;
R is a hydrogen or C(=O)C₁-C₈alkyl;
each of R₁, R₂ and R₃ independently is a hydrogen, optionally substituted methyl or
(CH₂)ₘ—CH₃;
R₄ is NR₅R₆, OR₅, OC(═O)R₇, C(═O)OR₅, C(═O)R₅, C(═O)NR₅R₆, halogen, 5 or 6-membered lactone, C₁₋C₈alkyl, C₂-C₈alkenyl, C₂-C₈alkynyl, aryl, glucosyl, wherein the 5 or 6-membered lactone, C₁₋C₈alkyl, C₂-C₈alkenyl, C₂-C₈alkynyl, aryl, and glucosyl are optionally substituted with one or more substituents selected from
NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁₋C₈alkyl, OR₅ or NR₅R₆;
m = 1-12; and
n=1-12; or a pharmaceutically acceptable salt, metabolite, solvate or prodrug thereof.

In some embodiments, there are provided pharmaceutical compositions comprising a therapeutically effective amount of a cyclohexenone compound having the structure:

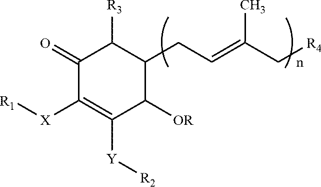

wherein each of X and Y independently is oxygen, NR₅ or sulfur;
R is a hydrogen or C(=O)C₁-C₈alkyl;
each of R₁, R₂ and R₃ independently is a hydrogen, optionally substituted methyl or
(CH₂)ₘ—CH₃;
R₄ is NR₅R₆, OR₅, OC(═O)R₇, C(═O)OR₅, C(═O)R₅, C(═O)NR₅R₆, halogen, 5 or 6-membered lactone, C₁₋C₈alkyl, C₂-C₈alkenyl, C₂-C₈alkynyl, aryl, glucosyl, wherein the 5 or 6-membered lactone, C₁₋C₈alkyl, C₂-C₈alkenyl, C₂-C₈alkynyl, aryl, and glucosyl are optionally substituted with one or more substituents selected from
NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁₋C₈alkyl, OR₅ or NR₅R₆;
m = 1-12; and
n=1-12; or a pharmaceutically acceptable salt, metabolite, solvate or prodrug thereof;
for use in treating or reducing symptoms of and/or preventing an RNA virus induced disease (such as a virus induced pneumonia) in a subject.

In some embodiments, there are provided uses of a therapeutically effective amount of a cyclohexenone compound having the structure

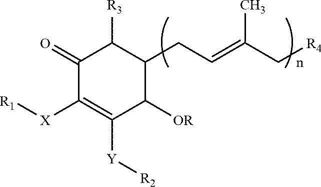

in the manufacture of a medicament for treating, reducing symptoms of and/or preventing an RNA virus induced disease (such as an RNA virus induced pneumonia) in a subject, wherein each of X and Y independently is oxygen, NR₅ or sulfur;
R is a hydrogen or C(=O)C₁-C₈alkyl;
each of R₁, R₂ and R₃ independently is a hydrogen, optionally substituted methyl or
(CH₂)ₘ—CH₃;
R₄ is NR₅R₆, OR₅, OC(═O)R₇, C(═O)OR₅, C(═O)R₅, C(═O)NR₅R₆, halogen, 5 or 6-membered lactone, C₁₋C₈alkyl, C₂-C₈alkenyl, C₂-C₈alkynyl, aryl, glucosyl, wherein the 5 or 6-membered lactone, C₁₋C₈alkyl, C₂-C₈alkenyl, C₂-C₈alkynyl, aryl, and glucosyl are optionally substituted with one or more substituents selected from
NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁₋C₈alkyl, OR₅ or NR₅R₆;
m = 1-12; and n=1-12; or a pharmaceutically acceptable salt, metabolite, solvate or prodrug thereof.

In some embodiments, said RNA virus induced disease is an RNA virus induced pneumonia, a coronavirus induced pneumonia, or a SARS-CoV-2 induced pneumonia, or the like. In certain embodiments, the RNA virus is a coronavirus. In some embodiments, the RNA virus induced disease is caused or induced by a coronaviridae infection. In some embodiments, In certain embodiments, said conronaviridae infection is caused by, or associated with alpha coronaviruses 229E (HCoV-229E), NL63 (HCoV-NL63, New Haven coronavirus), beta coronaviruses OC43 (HCoV-OC43), HKU1, MERS-CoV (the coronavirus responsible for Middle East Respiratory Syndrome), SARS-CoV (the coronavirus responsible for Severe Acute Respiratory Syndrome) or SARS-CoV-2 (the coronavirus responsible for Severe Acute Respiratory Syndrome, previously known as novel coronavirus in 2019, or 2019-nCoV), or the like. In certain embodiments, said coronaviridae infection is caused by or associated with severe acute respiratory syndrome coronavirus 2 (SARS-CoV-2). In some embodiments, said RNA virus induced disease is an RNA virus induced pneumonia. In certain embodiments, said coronaviridae infection is caused by or associated with severe acute respiratory syndrome coronavirus 2 (SARS-CoV-2). In some embodiments, the cyclohexenone compound reduces RNA virus concentration or prevents the RNA virus replication. In certain embodiments, the cyclohexenone compound reduces RNA virus concentration or prevents the RNA virus replication of alpha coronaviruses 229E (HCoV-229E), NL63 (HCoV-NL63, New Haven coronavirus), beta coronaviruses OC43 (HCoV-OC43), HKU1, MERS-CoV (the coronavirus responsible for Middle East Respiratory Syndrome), SARS-CoV (the coronavirus responsible for Severe Acute Respiratory Syndrome) or SARS-CoV-2 (the coronavirus responsible for Severe Acute Respiratory Syndrome, previously known as novel coronavirus in 2019, or 2019-nCoV), or the like. In some embodiments, the subject is human.

In some embodiments provide a method for treating, inhibiting and/or preventing a coronavirus-induced pneumonia in a subject in need thereof, comprising administering an effective amount of a cyclohexenone compound of the following formula (I) to said subject.

In some embodiments provide a method for treating, inhibiting and/or preventing an RNA virus replication (such as a coronavirus replication) in a subject in need thereof, comprising administering an effective amount of a cyclohexenone compound disclosed herein to a subject.

In some embodiments provide a method for reducing RNA virus concentration in a subject in need thereof, comprising administering an effective amount of a cyclohexenone compound disclosed herein to a subject.

In some embodiments provide a method for inhibiting and/or preventing an RNA virus infection in a subject in need thereof, comprising administering an effective amount of a cyclohexenone compound disclosed herein to a subject.

In some embodiments, the cyclohexenone compound having the structure

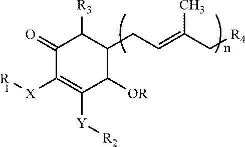

is prepared synthetically or semi-synthetically from any suitable starting material. In other embodiments, the cyclohexenone compound is prepared by fermentation, or the like. For example, Compounds 1, and 3-7 are isolated from organic solvent extracts. The non-limited exemplary compounds are illustrated below.

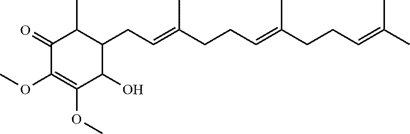 [SMILES: COC1=C(OC)C(O)C(C/C=C(\C)CC/C=C(\C)CCC=C(C)C)C(C)C1=O]

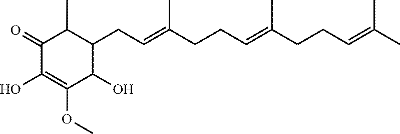 [SMILES: COC1=C(O)C(=O)C(C)C(C/C=C(\C)CC/C=C(\C)CCC=C(C)C)C1O]

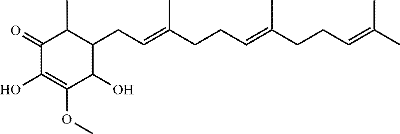 [SMILES: COC1=C(O)C(=O)C(C)C(C/C=C(\C)CC/C=C(\C)CCC=C(C)C)C1O]

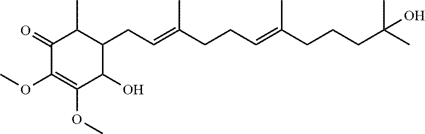 [SMILES: COC1=C(OC)C(O)C(C/C=C(\C)CC/C=C(\C)CCCC(C)(C)O)C(C)C1=O]

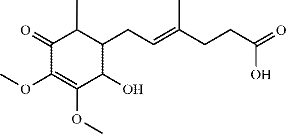 [SMILES: COC1=C(OC)C(O)C(C/C=C(\C)CCC(=O)O)C(C)C1=O]

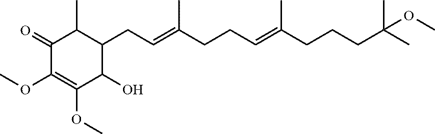

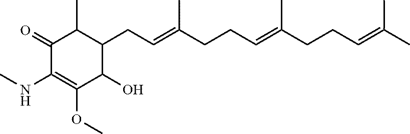 [SMILES: CNC1=C(OC)C(O)C(C/C=C(\C)CC/C=C(\C)CCC=C(C)C)C(C)C1=O]

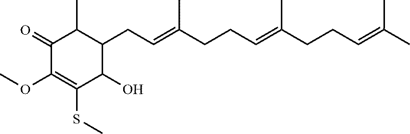 [SMILES: COC1=C(SC)C(O)C(C/C=C(\C)CC/C=C(\C)CCC=C(C)C)C(C)C1=O]

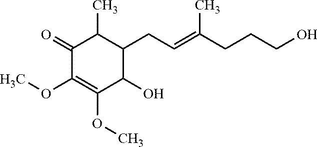 [SMILES: COC1=C(OC)C(O)C(C/C=C(\C)CCCO)C(C)C1=O]

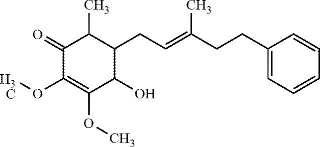 [SMILES: COC1=C(OC)C(O)C(C/C=C(\C)CCc2ccccc2)C(C)C1=O]

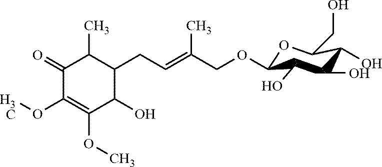

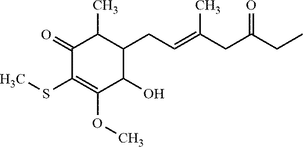 [SMILES: CCC(=O)C/C(C)=C/CC1C(C)C(=O)C(SC)=C(OC)C1O]

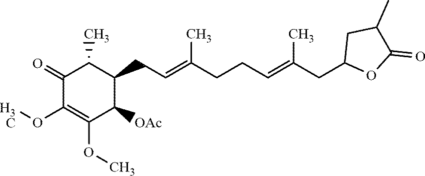 [SMILES: COC1=C(OC)[C@H](OC(C)=O)[C@H](C/C=C(\C)CC/C=C(\C)CC2CC(C)C(=O)O2)[C@@H](C)C1=O]

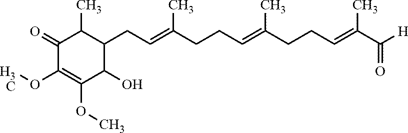 [SMILES: COC1=C(OC)C(O)C(C/C=C(\C)CC/C=C(\C)CC/C=C(\C)C=O)C(C)C1=O]

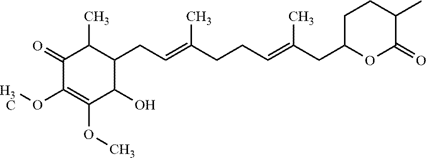

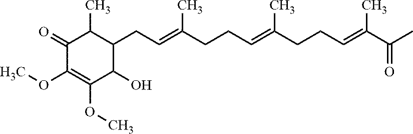 [SMILES: COC1=C(OC)C(O)C(C/C=C(\C)CC/C=C(\C)CC/C=C(\C)C(C)=O)C(C)C1=O]

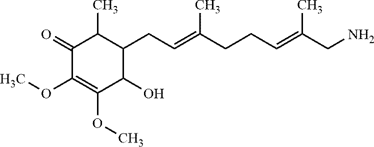 [SMILES: COC1=C(OC)C(O)C(C/C=C(\C)CC/C=C(\C)CN)C(C)C1=O]

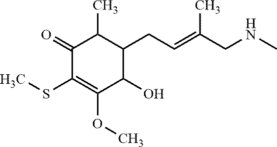

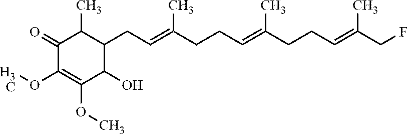 [SMILES: COC1=C(OC)C(O)C(C/C=C(\C)CC/C=C(\C)CC/C=C(\C)CF)C(C)C1=O]

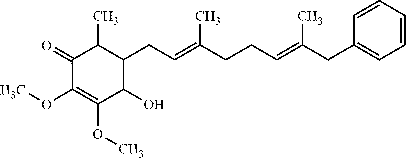 [SMILES: COC1=C(OC)C(O)C(C/C=C(\C)CC/C=C(\C)Cc2ccccc2)C(C)C1=O]

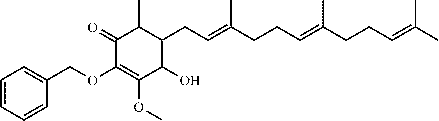 [SMILES: COC1=C(OCc2ccccc2)C(=O)C(C)C(C/C=C(\C)CC/C=C(\C)CCC=C(C)C)C1O]

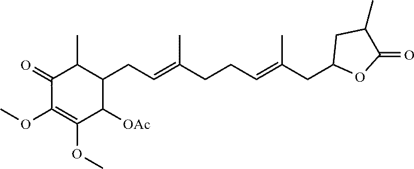 [SMILES: COC1=C(OC)C(OC(C)=O)C(C/C=C(\C)CC/C=C(\C)CC2CC(C)C(=O)O2)C(C)C1=O]

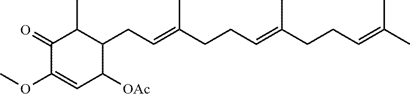 [SMILES: COC1=CC(OC(C)=O)C(C/C=C(\C)CC/C=C(\C)CCC=C(C)C)C(C)C1=O]

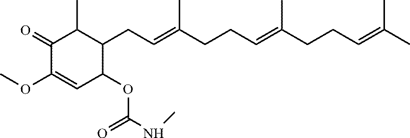 [SMILES: CNC(=O)OC1C=C(OC)C(=O)C(C)C1C/C=C(\C)CC/C=C(\C)CCC=C(C)C]

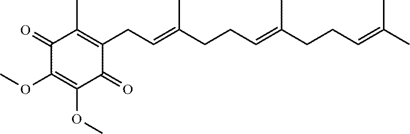 [SMILES: COC1=C(OC)C(=O)C(C/C=C(\C)CC/C=C(\C)CCC=C(C)C)=C(C)C1=O]

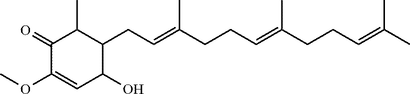 [SMILES: COC1=CC(O)C(C/C=C(\C)CC/C=C(\C)CCC=C(C)C)C(C)C1=O]

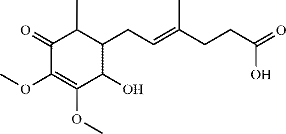 [SMILES: COC1=C(OC)C(O)C(C/C=C(\C)CCC(=O)O)C(C)C1=O]

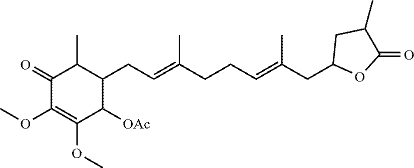 [SMILES: COC1=C(OC)C(OC(C)=O)C(C/C=C(\C)CC/C=C(\C)CC2CC(C)C(=O)O2)C(C)C1=O]

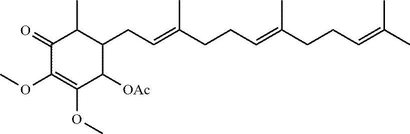 [SMILES: COC1=C(OC)C(OC(C)=O)C(C/C=C(\C)CC/C=C(\C)CCC=C(C)C)C(C)C1=O]

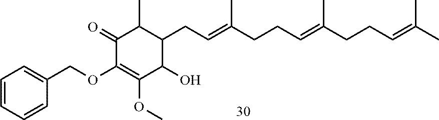

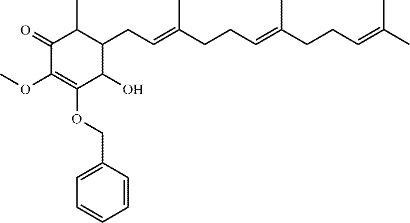 [SMILES: COC1=C(OCc2ccccc2)C(O)C(C/C=C(\C)CC/C=C(\C)CCC=C(C)C)C(C)C1=O]

In other embodiments, the cyclohexenone compound having the structure

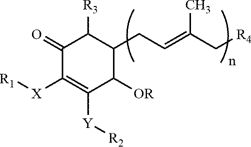

is isolated from the organic solvent extracts of Antrodia camphorata. In some embodiments, the organic solvent is selected from alcohols (e.g., methanol, ethanol, propanol, or the like), esters (e.g., methyl acetate, ethyl acetate, or the like), alkanes (e.g., pentane, hexane, heptane, or the like), halogenated alkanes (e.g., chloromethane, chloroethane, chloroform, methylene chloride, and the like), and the like. For example, exemplary Compounds 1-7 are isolated from organic solvent extracts. In certain embodiments, the organic solvent is alcohol. In certain embodiments, the alcohol is ethanol. In some embodiments, the cyclohexenone compound is isolated from the aqueous extracts of Antrodia camphorata. In certain embodiments, the cyclohexenone compounds disclosed herein are prepared synthetically or semi-synthetically.

In some embodiments, each of X and Y independently is oxygen, or sulfur. It is known in the art that a compound where each X and Y independently is sulfur can be prepared similarly or by the same route of the compound where each of X and Y independently is oxygen, because oxygen and sulfur share similar chemical property in a structure. In some embodiments, by a proper protecting group, the compound where each of X and Y independently is NR₅ can be prepared by the similar route of a compound where each of X and Y independently is oxygen or sulfur.

In some embodiments, R is a hydrogen, C(═O)C₃H₈, C(═O)C₂H₅, or C(═O)CH₃. In some embodiments, R₁ is a hydrogen, methyl, ethyl, propyl, butyl, pentyl or hexyl. In certain embodiments, R₁ is a hydrogen or methyl. In some embodiments, R₂ is a hydrogen, methyl, ethyl, propyl, butyl, pentyl or hexyl. In certain embodiments, R₂ is a hydrogen or methyl. In some embodiments, R₃ is a hydrogen, methyl, ethyl, propyl, butyl, pentyl or hexyl. In some embodiments, R₄ is halogen, NH₂, NHCH₃, N(CH₃)₂, OCH₃, OC₂H₅, C(═O)CH₃, C(═O)C₂H₅, C(═O)OCH₃, C(═O)OC₂H₅, C(═O)NHCH₃, C(═O)NHC₂H₅, C(═O)NH₂, OC(═O)CH₃, OC(═O)C₂H₅, OC(═O)OCH₃, OC(═O)OC₂H₅, OC(═O)NHCH₃, OC(═O)NHC₂H₅, or OC(═O)NH₂. In some embodiments, R₄ is C₂H₅C(CH₃)₂OH, C₂H₅C(CH₃)₂OCH₃, CH₂COOH, C₂H₅COOH, CH₂OH, C₂H₅OH, CH₂Ph, C₂H₅Ph, CH₂CH═C(CH₃)(CHO), CH₂CH═C(CH₃)(C(═O)CH₃), 5 or 6-membered lactone, C₂-C₈alkenyl, C₂-C₈alkynyl, aryl, and glucosyl, wherein 5 or 6-membered lactone, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl. In certain embodiments, R₄ is 5 or 6-membered lactone, C₂-C₈alkenyl, C₂-C₈alkynyl, aryl, and glucosyl, optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl. In certain embodiments, R₄ is CH₂CH═C(CH₃)₂. In certain embodiments, the compound is

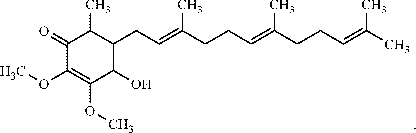

### Certain Pharmaceutical and Medical Terminology

Unless otherwise stated, the following terms used in this application, including the specification and claims, have the definitions given below. It must be noted that, as used in the specification and the appended claims, the singular forms “a,” “an” and “the” include plural referents unless the context clearly dictates otherwise. Unless otherwise indicated, conventional methods of mass spectroscopy, NMR, HPLC, protein chemistry, biochemistry, recombinant DNA techniques and pharmacology are employed. In this application, the use of “or” or “and” means “and/or” unless stated otherwise. Furthermore, use of the term “including” as well as other forms, such as “include”, “includes,” and “included,” is not limiting. The section headings used herein are for organizational purposes only and are not to be construed as limiting the subject matter described.

An “alkyl” group refers to an aliphatic hydrocarbon group. The alkyl group may be a saturated alkyl group (which means that it does not contain any carbon-carbon double bonds or carbon-carbon triple bonds) or the alkyl group may be an unsaturated alkyl group (which means that it contains at least one carbon-carbon double bonds or carbon-carbon triple bond). The alkyl moiety, whether saturated or unsaturated, may be branched, or straight chain.

The “alkyl” group may have 1 to 12 carbon atoms (whenever it appears herein, a numerical range such as “1 to 12 refers to each integer in the given range; e.g., “1 to 12 carbon atoms” means that the alkyl group may consist of 1 carbon atom, 2 carbon atoms, 3 carbon atoms, etc., up to and including 12 carbon atoms, although the present definition also covers the occurrence of the term “alkyl” where no numerical range is designated). The alkyl group of the compounds described herein may be designated as “C₁-C₈ alkyl” or similar designations. By way of example only, “C₁-C₈ alkyl” indicates that there are one, two, three, four, five, six, seven or eight carbon atoms in the alkyl chain. In one aspect the alkyl is selected from the group consisting of methyl, ethyl, propyl, iso-propyl, n-butyl, iso-butyl, sec-butyl, and t-butyl. Typical alkyl groups include, but are in no way limited to, methyl, ethyl, propyl, isopropyl, butyl, isobutyl, sec-butyl, tertiary butyl, pentyl, neopentyl, hexyl, allyl, but-2-enyl, but-3-enyl, cyclopropylmethyl, cyclobutylmethyl, cyclopentylmethyl, cyclohexylmethyl, and the like. In one aspect, an alkyl is a C₁-C₈ alkyl.

The term “alkylene” refers to a divalent alkyl radical. Any of the above mentioned monovalent alkyl groups may be an alkylene by abstraction of a second hydrogen atom from the alkyl. In one aspect, an alkylene is a C₁-C₁₂alkylene. In another aspect, an alkylene is a C₁-C₈alkylene. Typical alkylene groups include, but are not limited to, —CH₂—, —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, —CH₂(CH₂)₃CH₂—, and the like.

As used herein, the term “aryl” refers to an aromatic ring wherein each of the atoms forming the ring is a carbon atom. Aryl rings are formed by five, six, seven, eight, nine, or more than nine carbon atoms. Aryl groups are optionally substituted. In one aspect, an aryl is a phenyl or a naphthalenyl. In one aspect, an aryl is a phenyl. In one aspect, an aryl is a C₆-C₁₀aryl. Depending on the structure, an aryl group can be a monoradical or a diradical (i.e., an arylene group). In one aspect, an arylene is a C₆-C₁₀ arylene. Exemplary arylenes include, but are not limited to, phenyl-1,2-ene, phenyl-1,3-ene, and phenyl-1,4-ene.

The term “aromatic” refers to a planar ring having a delocalized π-electron system containing 4n+2 π electrons, where n is an integer. Aromatic rings can be formed from five, six, seven, eight, nine, ten, or more than ten atoms. Aromatics are optionally substituted. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “halo” or, alternatively, “halogen” or “halide” means fluoro, chloro, bromo or iodo.

The term “lactone” refers to a cyclic ester which can be seen as the condensation product of an alcohol group —OH and a carboxylic acid group —COOH in the same molecule. It is characterized by a closed ring consisting of two or more carbon atoms and a single oxygen atom, with a ketone group ═O in one of the carbons adjacent to the other oxygen.

The term “heterocycle” or “heterocyclic” refers to heteroaromatic rings (also known as heteroaryls) and heterocycloalkyl rings (also known as heteroalicyclic groups) containing one to four heteroatoms in the ring(s), where each heteroatom in the ring(s) is selected from O, S and N, wherein each heterocyclic group has from 4 to 10 atoms in its ring system, and with the proviso that the any ring does not contain two adjacent O or S atoms. Non-aromatic heterocyclic groups (also known as heterocycloalkyls) include groups having only 3 atoms in their ring system, but aromatic heterocyclic groups must have at least 5 atoms in their ring system. The heterocyclic groups include benzo-fused ring systems. An example of a 3-membered heterocyclic group is aziridinyl. An example of a 4-membered heterocyclic group is azetidinyl. An example of a 5-membered heterocyclic group is thiazolyl. An example of a 6-membered heterocyclic group is pyridyl, and an example of a 10-membered heterocyclic group is quinolinyl. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups may be C-attached or N-attached where such is possible. For instance, a group derived from pyrrole may be pyrrol-1-yl (N-attached) or pyrrol-3-yl (C-attached). Further, a group derived from imidazole may be imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles may be substituted with one or two oxo (=O) moieties, such as pyrrolidin-2-one.

The term “alkenyl” as used herein, means a straight, branched chain, or cyclic (in which case, it would also be known as a “cycloalkenyl”) hydrocarbon containing from 2-10 carbons and containing at least one carbon-carbon double bond formed by the removal of two hydrogens. In some embodiments, depending on the structure, an alkenyl group is a monoradical or a diradical (i.e., an alkenylene group). In some embodiments, alkenyl groups are optionally substituted. Illustrative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-cecenyl.

The term “alkynyl” as used herein, means a straight, branched chain, or cyclic (in which case, it would also be known as a “cycloalkynyl”) hydrocarbon containing from 2-10 carbons and containing at least one carbon-carbon triple bond formed by the removal of four hydrogens. In some embodiments, depending on the structure, an alkynyl group is a monoradical or a diradical (i.e., an alkynylene group). In some embodiments, alkynyl groups are optionally substituted. Illustrative examples of alkynyl include, but are not limited to, ethynyl, propynyl, butynyl, pentynyl, hexynyl, heptynyl, and the like.

The term “alkoxy” as used herein, means an alkyl group, as defined herein, appended to the parent molecular moiety through an oxygen atom. Illustrative examples of alkoxy include, but are not limited to, methoxy, ethoxy, propoxy, 2-propoxy, butoxy, tert-butoxy, pentyloxy, and hexyloxy.

The term “cycloalkyl” as used herein, means a monocyclic or polycyclic radical that contains only carbon and hydrogen, and includes those that are saturated, partially unsaturated, or fully unsaturated. Cycloalkyl groups include groups having from 3 to 10 ring atoms. Representative examples of cyclic include but are not limited to, the following moieties:

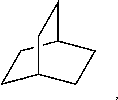

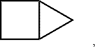

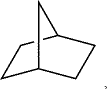

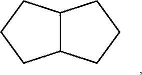

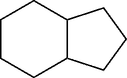

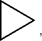

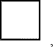

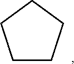

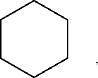

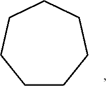

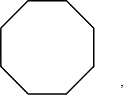

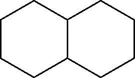

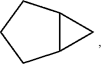

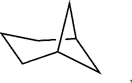

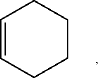

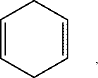

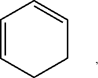

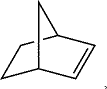

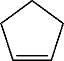

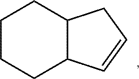

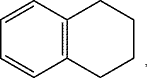

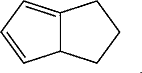

In some embodiments, depending on the structure, a cycloalkyl group is a monoradical or a diradical (e.g., a cycloalkylene group).

The terms “haloalkyl,” “haloalkenyl,” “haloalkynyl” and “haloalkoxy” as used herein, include alkyl, alkenyl, alkynyl and alkoxy structures in which at least one hydrogen is replaced with a halogen atom. In certain embodiments in which two or more hydrogen atoms are replaced with halogen atoms, the halogen atoms are all the same as one another. In other embodiments in which two or more hydrogen atoms are replaced with halogen atoms, the halogen atoms are not all the same as one another. The terms “fluoroalkyl” and “fluoroalkoxy” include haloalkyl and haloalkoxy groups, respectively, in which the halo is fluorine. In certain embodiments, haloalkyls are optionally substituted.

The term “glucosyl” as used herein, include D- or L-form glucosyl groups, in which the glucosyl group is attached via any hydroxyl group on the glucose ring.

The term “acceptable” with respect to a formulation, composition or ingredient, as used herein, means having no persistent detrimental effect on the general health of the subj ect being treated.

Antrodia is a genus of fungi in the family Meripilaceae. Antrodia species have fruiting bodies that typically lie flat or spread out on the growing surface, with the hymenium exposed to the outside; the edges may be turned so as to form narrow brackets. Most species are found in temperate and boreal forests, and cause brown rot.

Antrodia camphorata, also known as stout camphor fungus, Ganoderma camphoratum, is a species of Antrodia fungi, that is endemic to Taiwan, where it grows only on the endemic tree Cinnamomum kanehirae, causing a brown heart rot. This unique mushroom of Taiwan has been used as a traditional medicine for protection of different disease conditions.

It is known in the art that the active ingredients isolated from the different parts of Antrodia camphorata vary by different cultural medium, and methods. For example, certain cyclohexenone compounds disclosed herein can only be isolated from the unique solid state fermentation process to cultivate Antrodia camphorata which is different from other known methods.

The term “carrier,” as used herein, refers to relatively nontoxic chemical compounds or agents that facilitate the incorporation of a compound into cells or tissues.

The terms “co-administration” or the like, as used herein, are meant to encompass administration of the selected therapeutic agents to a single patient, and are intended to include treatment regimens in which the agents are administered by the same or different route of administration or at the same or different time.

The term “diluent” refers to chemical compounds that are used to dilute the compound of interest prior to delivery. Diluents can also be used to stabilize compounds because they can provide a more stable environment. Salts dissolved in buffered solutions (which also can provide pH control or maintenance) are utilized as diluents in the art, including, but not limited to a phosphate buffered saline solution.

The terms “effective amount” or “therapeutically effective amount,” as used herein, refer to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

A “metabolite” of a compound disclosed herein is a derivative of that compound that is formed when the compound is metabolized. The term “active metabolite” refers to a biologically active derivative of a compound that is formed when the compound is metabolized. The term “metabolized,” as used herein, refers to the sum of the processes (including, but not limited to, hydrolysis reactions and reactions catalyzed by enzymes) by which a particular substance is changed by an organism. Thus, enzymes may produce specific structural alterations to a compound. For example, cytochrome P450 catalyzes a variety of oxidative and reductive reactions while uridine diphosphate glucuronyltransferases catalyze the transfer of an activated glucuronic-acid molecule to aromatic alcohols, aliphatic alcohols, carboxylic acids, amines and free sulphydryl groups. Metabolites of the compounds disclosed herein are optionally identified either by administration of compounds to a host and analysis of tissue samples from the host, or by incubation of compounds with hepatic cells in vitro and analysis of the resulting compounds.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound (i.e., a cyclohexenone compound described herein) and a co-agent, are both administered to a patient simultaneously in the form of a single entity or dosage. The term “non-fixed combination” means that the active ingredients, e.g. a compound (i.e., a cyclohexenone compound described herein) and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The term “pharmaceutical composition” refers to a mixture of a compound (i.e., a cyclohexenone compound described herein) with other chemical components, such as carriers, stabilizers, diluents, dispersing agents, suspending agents, thickening agents, and/or excipients. The pharmaceutical composition facilitates administration of the compound to an organism. Multiple techniques of administering a compound exist in the art including, but not limited to: intravenous, oral, aerosol, parenteral, ophthalmic, pulmonary and topical administration.

The term “subject” or “patient” encompasses mammals and birds. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one embodiment, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically. Specially, the term “treat,” “treatment” or “treating” means reducing the frequency, extent, severity and/or duration with which symptoms of coronavirus-induced disease are experienced by a subject (e.g., a patient).

The term “prevent,” “prevention” or “preventing” means inhibition, risk reduction, reducing the onset of or the averting of symptoms associated with coronavirus-induced disease.

### Routes of Administration and Dosage

Suitable routes of administration include, but are not limited to, oral, intravenous, rectal, aerosol, parenteral, ophthalmic, pulmonary, transmucosal, transdermal, vaginal, otic, nasal, and topical administration. In addition, by way of example only, parenteral delivery includes intramuscular, subcutaneous, intravenous, intramedullary injections, as well as intrathecal, direct intraventricular, intraperitoneal, intralymphatic, and intranasal injections.

In certain embodiments, a compound as described herein is administered in a local rather than systemic manner, for example, via injection of the compound directly into an organ, often in a depot preparation or sustained release formulation. In specific embodiments, long acting formulations are administered by implantation (for example subcutaneously or intramuscularly) or by intramuscular injection. Furthermore, in other embodiments, the drug is delivered in a targeted drug delivery system, for example, in a liposome coated with organ-specific antibody. In such embodiments, the liposomes are targeted to and taken up selectively by the organ. In yet other embodiments, the compound as described herein is provided in the form of a rapid release formulation, in the form of an extended release formulation, or in the form of an intermediate release formulation. In yet other embodiments, the compound described herein is administered topically.

In some embodiments, the cyclohexenone compound, or a pharmaceutically acceptable salt, metabolite, solvate or prodrug thereof, is administered parenterally or intravenously. In other embodiments, the cyclohexenone compound, or a pharmaceutically acceptable salt, metabolite, solvate or prodrug thereof, is administered by injection. In some embodiments, the cyclohexenone compound, or a pharmaceutically acceptable salt, metabolite, solvate or prodrug thereof, is administered orally.

In the case wherein the patient’s condition does not improve, upon the doctor’s discretion the administration of the compounds may be administered chronically, that is, for an extended period of time, including throughout the duration of the patient’s life in order to ameliorate or otherwise control or limit the symptoms of the patient’s disease or condition. In the case wherein the patient’s status does improve, upon the doctor’s discretion the administration of the compounds may be given continuously or temporarily suspended for a certain length of time (i.e., a “drug holiday”).

The foregoing ranges are merely suggestive, as the number of variables in regard to an individual treatment regime is large, and considerable excursions from these recommended values are not uncommon. Such dosages may be altered depending on a number of variables, not limited to the activity of the compound used, the disease or condition to be treated, the mode of administration, the requirements of the individual subject, the severity of the disease or condition being treated, and the judgment of the practitioner.

Toxicity and therapeutic efficacy of such therapeutic regimens can be determined by standard pharmaceutical procedures in cell cultures or experimental animals, including, but not limited to, for determining the LD₅₀ (the dose lethal to 50% of the population) and the ED₅₀ (the dose therapeutically effective in 50% of the population). The dose ratio between the toxic and therapeutic effects is the therapeutic index and it can be expressed as the ratio between LD₅₀ and ED₅₀. Compounds exhibiting high therapeutic indices are preferred. The data obtained from cell culture assays and animal studies can be used in formulating a range of dosage for use in human. The dosage of such compounds lies preferably within a range of circulating concentrations that include the ED₅₀ with minimal toxicity. The dosage may vary within this range depending upon the dosage form employed and the route of administration utilized.

It is understood that in some embodiments, the dosage regimen to treat, prevent, or ameliorate the condition(s) for which relief is sought, is modified in accordance with a variety of factors. These factors include the disorder from which the subject suffers, as well as the age, weight, sex, diet, and medical condition of the subject. Thus, in other embodiments, the dosage regimen actually employed varies widely and therefore deviates from the dosage regimens set forth herein.

### Pharmaceutical Formulation

In some embodiments provide pharmaceutical compositions comprising a therapeutically effective amount of a cyclohexenone compound having the structure:

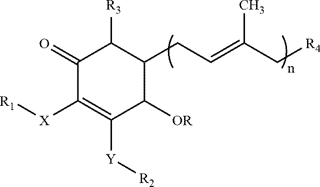

wherein each of X and Y independently is oxygen, NR₅ or sulfur;
R is a hydrogen or C(═O)C₁-C₈alkyl;
each of R₁, R₂ and R₃ independently is a hydrogen, optionally substituted methyl or
(CH₂)ₘ—CH₃;
R₄ is NR₅R₆, OR₅, OC(═O)R₇, C(═O)OR₅, C(═O)R₅, C(═O)NR₅R₆, halogen, 5 or 6-membered lactone, C₁₋C₈alkyl, C₂-C₈alkenyl, C₂-C₈alkynyl, aryl, glucosyl, wherein the 5 or 6-membered lactone, C₁₋C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁₋C₈alkyl, OR₅ or NR₅R₆;
m = 1-12; and n=1-12; or a pharmaceutically acceptable salt, metabolite, solvate or prodrug thereof; and a pharmaceutically acceptable excipient.

In some embodiments, the cyclohexenone compounds of the pharmaceutical compositions have the structure:

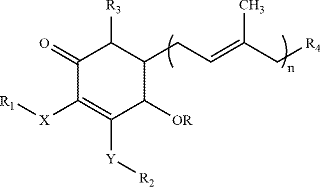

wherein each of X and Y independently is oxygen, NR₅ or sulfur;
R is a hydrogen or C(═O)C₁-C₈alkyl;
each of R₁, R₂ and R₃ independently is a hydrogen, optionally substituted methyl or
(CH₂)ₘ-CH₃;
R₄ is NR₅R₆, OR₅, OC(═O)R₇, C(═O)OR₅, C(═O)R₅, C(═O)NR₅R₆, halogen, 5 or 6-membered lactone, C₁₋C₈alkyl, C₂-C₈alkenyl, C₂-C₈alkynyl, aryl, glucosyl, wherein the 5 or 6-membered lactone, C₁₋C₈alkyl, C₂-C₈alkenyl, C₂-C₈alkynyl, aryl, and glucosyl are optionally substituted with one or more substituents selected from
NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl;
each of R₅ and R₆ is independently a hydrogen or C₁-C₈alkyl;
R₇ is a C₁₋C₈alkyl, OR₅ or NR₅R₆;
m = 1-12; and n= 1-12; or a pharmaceutically acceptable salt, metabolite, solvate or prodrug thereof.

In some embodiments, R is a hydrogen, C(═O)C₃H₈, C(═O)C₂H₅, or C(═O)CH₃. In some embodiments, each of R₁, R₂ and R₃ independently is a hydrogen, methyl, ethyl, propyl, butyl, pentyl hexyl, heptyl, or octyl.. In certain embodiments, R₁ is a hydrogen or methyl. In certain embodiments, R₂ is a hydrogen, methyl, ethyl, propyl, butyl, pentyl or hexyl. In certain embodiments, R₃ is a hydrogen, methyl, ethyl, propyl, butyl, pentyl or hexyl. In some embodiments, R₄ is halogen, NH₂, NHCH₃, N(CH₃)₂, OCH₃, OC₂H₅, C(═O)CH₃, C(═O)C₂H₅, C(═O)OCH₃, C(═O)OC₂H₅, C(═O)NHCH₃, C(═O)NHC₂H₅, C(═O)NH₂, OC(═O)CH₃, OC(═O)C₂H₅, OC(═O)OCH₃, OC(═O)OC₂H₅, OC(═O)NHCH₃, OC(═O)NHC₂H₅, or OC(═O)NH₂. In certain embodiments, R₄ is C₂H₅C(CH₃)₂OH, C₂H₅C(CH₃)₂OCH₃, CH₂COOH, C₂H₅COOH, CH₂OH, C₂H₅OH, CH₂Ph, C₂H₅Ph, CH₂CH═C(CH₃)(CHO), CH₂CH═C(CH₃)(C(═O)CH₃), 5 or 6-membered lactone, aryl, or glucosyl, wherein the 5 or 6-membered lactone, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl. In certain embodiments, R₄ is CH₂COOH, C₂H₅COOH, CH₂OH, C₂H₅OH, CH₂Ph, C₂H₅Ph, CH₂CH═C(CH₃)(CHO), CH₂CH═C(CH₃)(C(═O)CH₃), 5 or 6-membered lactone, aryl, or glucosyl, wherein 5 or 6-membered lactone, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl. In certain embodiments, R₄ is 5 or 6-membered lactone, aryl, or glucosyl, optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl.

In certain embodiments, the compound is selected from group consisting of

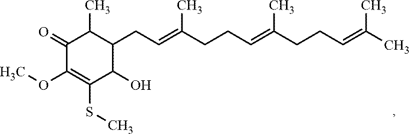

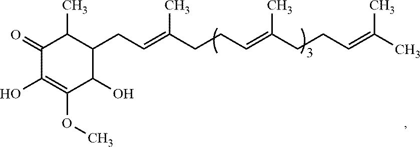

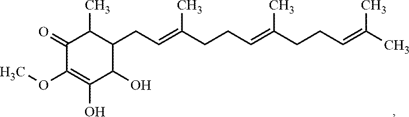

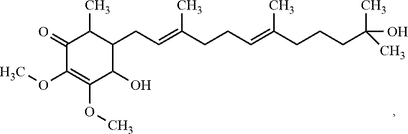

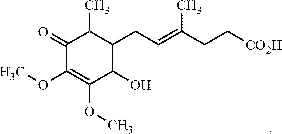

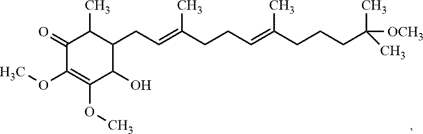

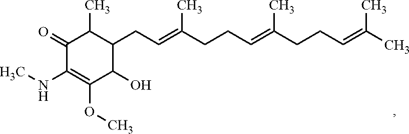

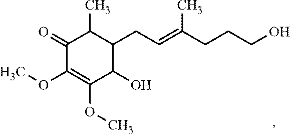

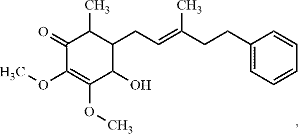

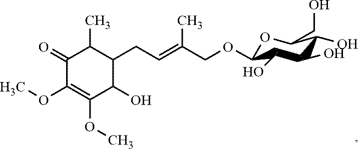

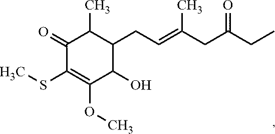

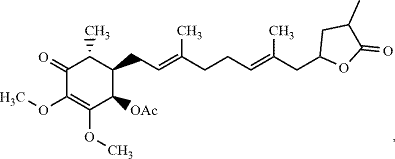

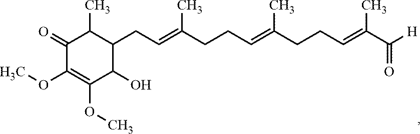

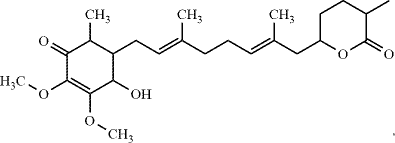

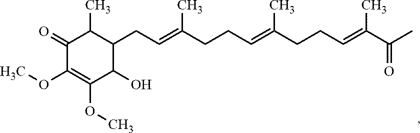

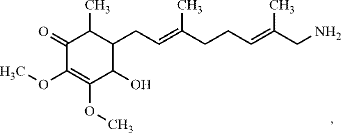

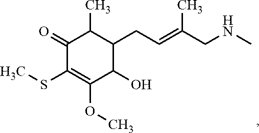

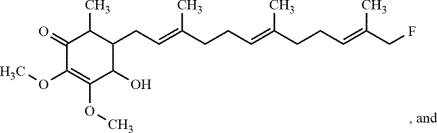

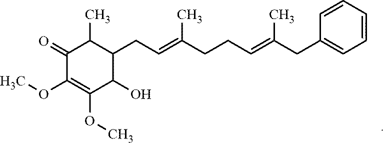

In certain embodiments, the compound is selected from group consisting of

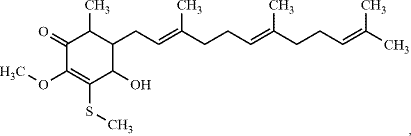

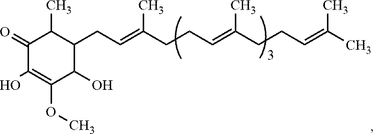

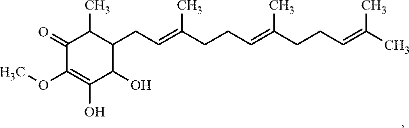

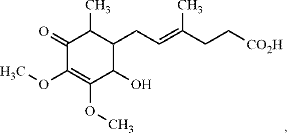

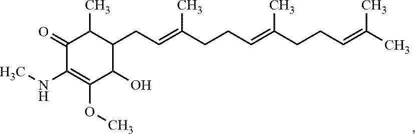

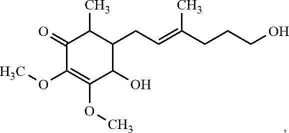

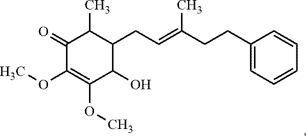

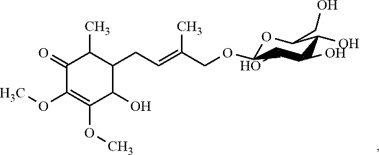

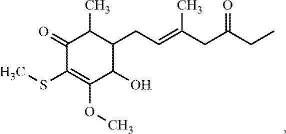

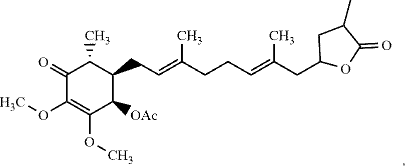

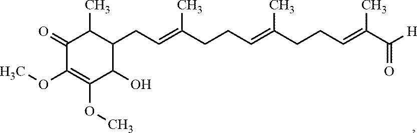

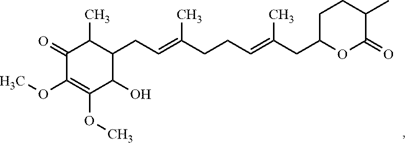

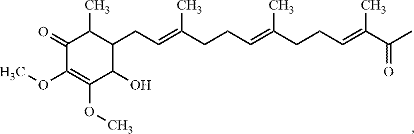

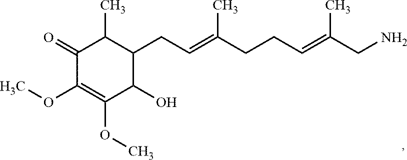

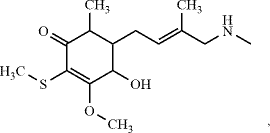

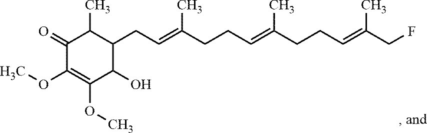

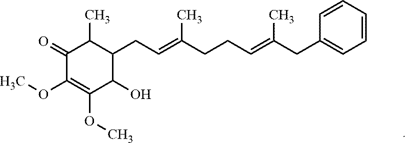

In some embodiments, the compounds described herein are formulated into pharmaceutical compositions. In specific embodiments, pharmaceutical compositions are formulated in a conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients are used as suitable to formulate the pharmaceutical compositions described herein: Remington: The Science and Practice of Pharmacy, Nineteenth Ed (Easton, Pa.: Mack Publishing Company, 1995); Hoover, John E., Remington’s Pharmaceutical Sciences, Mack Publishing Co., Easton, Pennsylvania 1975; Liberman, H.A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999).

Provided herein are pharmaceutical compositions comprising a compound (i.e., a cyclohexenone compound described herein) and a pharmaceutically acceptable diluent(s), excipient(s), or carrier(s). In certain embodiments, the compounds described are administered as pharmaceutical compositions in which a compound (i.e., a cyclohexenone compound described herein) is mixed with other active ingredients, as in combination therapy. Encompassed herein are all combinations of actives set forth in the combination therapies section below and throughout this disclosure. In specific embodiments, the pharmaceutical compositions include one or more compounds (i.e., a cyclohexenone compound described herein).

A pharmaceutical composition, as used herein, refers to a mixture of a compound (i.e., a cyclohexenone compound described herein) with other chemical components, such as carriers, stabilizers, diluents, dispersing agents, suspending agents, thickening agents, and/or excipients. In certain embodiments, the pharmaceutical composition facilitates administration of the compound to an organism. In some embodiments, practicing the methods of treatment or use provided herein, therapeutically effective amounts of compounds (i.e., a cyclohexenone compound described herein) are administered in a pharmaceutical composition to a mammal having a disease or condition to be treated. In specific embodiments, the mammal is a human. In certain embodiments, therapeutically effective amounts vary depending on the severity of the disease, the age and relative health of the subject, the potency of the compound used and other factors. The compounds described herein are used singly or in combination with one or more therapeutic agents as components of mixtures.

In one embodiment, a compound (i.e., a cyclohexenone compound described herein) is formulated in an aqueous solution. In specific embodiments, the aqueous solution is selected from, by way of example only, a physiologically compatible buffer, such as Hank’s solution, Ringer’s solution, or physiological saline buffer. In other embodiments, a compound (i.e., a cyclohexenone compound described herein) is formulated for transmucosal administration. In specific embodiments, transmucosal formulations include penetrants that are appropriate to the barrier to be permeated. In still other embodiments wherein the compounds described herein are formulated for other parenteral injections, appropriate formulations include aqueous or nonaqueous solutions. In specific embodiments, such solutions include physiologically compatible buffers and/or excipients.

In another embodiment, compounds described herein are formulated for oral administration. Compounds described herein, including a compound (i.e., a cyclohexenone compound described herein), are formulated by combining the active compounds with, e.g., pharmaceutically acceptable carriers or excipients. In various embodiments, the compounds described herein are formulated in oral dosage forms that include, by way of example only, tablets, powders, pills, dragees, capsules, liquids, gels, syrups, elixirs, slurries, suspensions and the like.

In certain embodiments, pharmaceutical preparations for oral use are obtained by mixing one or more solid excipients with one or more of the compounds described herein, optionally grinding the resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as: for example, maize starch, wheat starch, rice starch, potato starch, gelatin, gum tragacanth, methylcellulose, microcrystalline cellulose, hydroxypropylmethylcellulose, sodium carboxymethylcellulose; or others such as: polyvinylpyrrolidone (PVP or povidone) or calcium phosphate. In specific embodiments, disintegrating agents are optionally added. Disintegrating agents include, by way of example only, cross-linked croscarmellose sodium, polyvinylpyrrolidone, agar, or alginic acid or a salt thereof such as sodium alginate.

In one embodiment, dosage forms, such as dragee cores and tablets, are provided with one or more suitable coating. In specific embodiments, concentrated sugar solutions are used for coating the dosage form. The sugar solutions, optionally contain additional components, such as by way of example only, gum arabic, talc, polyvinylpyrrolidone, carbopol gel, polyethylene glycol, and/or titanium dioxide, lacquer solutions, and suitable organic solvents or solvent mixtures. Dyestuffs and/or pigments are also optionally added to the coatings for identification purposes. Additionally, the dyestuffs and/or pigments are optionally utilized to characterize different combinations of active compound doses.

In certain embodiments, therapeutically effective amounts of at least one of the compounds described herein are formulated into other oral dosage forms. Oral dosage forms include push-fit capsules made of gelatin, as well as soft, sealed capsules made of gelatin and a plasticizer, such as glycerol or sorbitol. In specific embodiments, push-fit capsules contain the active ingredients in admixture with one or more filler. Fillers include, by way of example only, lactose, binders such as starches, and/or lubricants such as talc or magnesium stearate and, optionally, stabilizers. In other embodiments, soft capsules, contain one or more active compound that is dissolved or suspended in a suitable liquid. Suitable liquids include, by way of example only, one or more fatty oil, liquid paraffin, or liquid polyethylene glycol. In addition, stabilizers are optionally added.

In other embodiments, therapeutically effective amounts of at least one of the compounds described herein are formulated for buccal or sublingual administration. Formulations suitable for buccal or sublingual administration include, by way of example only, tablets, lozenges, or gels. In still other embodiments, the compounds described herein are formulated for parental injection, including formulations suitable for bolus injection or continuous infusion. In specific embodiments, formulations for injection are presented in unit dosage form (e.g., in ampoules) or in multi-dose containers. Preservatives are, optionally, added to the injection formulations. In still other embodiments, the pharmaceutical compositions of a compound (i.e., a cyclohexenone compound described herein) are formulated in a form suitable for parenteral injection as a sterile suspensions, solutions or emulsions in oily or aqueous vehicles. Parenteral injection formulations optionally contain formulatory agents such as suspending, stabilizing and/or dispersing agents. In specific embodiments, pharmaceutical formulations for parenteral administration include aqueous solutions of the active compounds in water-soluble form. In additional embodiments, suspensions of the active compounds are prepared as appropriate oily injection suspensions. Suitable lipophilic solvents or vehicles for use in the pharmaceutical compositions described herein include, by way of example only, fatty oils such as sesame oil, or synthetic fatty acid esters, such as ethyl oleate or triglycerides, or liposomes. In certain specific embodiments, aqueous injection suspensions contain substances which increase the viscosity of the suspension, such as sodium carboxymethyl cellulose, sorbitol, or dextran. Optionally, the suspension contains suitable stabilizers or agents which increase the solubility of the compounds to allow for the preparation of highly concentrated solutions. Alternatively, in other embodiments, the active ingredient is in powder form for constitution with a suitable vehicle, e.g., sterile pyrogen-free water, before use.

In one aspect, compounds (i.e., cyclohexenone compounds described herein) are prepared as solutions for parenteral injection as described herein or known in the art and administered with an automatic injector. Automatic injectors, such as those disclosed in U.S. Pat. Nos. 4,031,893, 5,358,489; 5,540,664; 5,665,071, 5,695,472 and WO/2005/087297 (each of which are incorporated herein by reference for such disclosure) are known. In general, all automatic injectors contain a volume of solution that includes a compound (i.e., a cyclohexenone compound described herein) to be injected. In general, automatic injectors include a reservoir for holding the solution, which is in fluid communication with a needle for delivering the drug, as well as a mechanism for automatically deploying the needle, inserting the needle into the patient and delivering the dose into the patient. Exemplary injectors provide about 0.3 mL, 0.6 mL, 1.0 mL or other suitable volume of solution at about a concentration of 0.5 mg to 50 mg of a compound (i.e., a cyclohexenone compound described herein) per 1 mL of solution. Each injector is capable of delivering only one dose of the compound.

In still other embodiments, the compounds (i.e., cyclohexenone compounds described herein) are administered topically. The compounds described herein are formulated into a variety of topically administrable compositions, such as solutions, suspensions, lotions, gels, pastes, medicated sticks, balms, creams or ointments. Such pharmaceutical compositions optionally contain solubilizers, stabilizers, tonicity enhancing agents, buffers and preservatives.

In yet other embodiments, the compounds (i.e., cyclohexenone compounds described herein) are formulated for transdermal administration. In specific embodiments, transdermal formulations employ transdermal delivery devices and transdermal delivery patches and can be lipophilic emulsions or buffered, aqueous solutions, dissolved and/or dispersed in a polymer or an adhesive. In various embodiments, such patches are constructed for continuous, pulsatile, or on demand delivery of pharmaceutical agents. In additional embodiments, the transdermal delivery of a compound (i.e., a cyclohexenone compound described herein) is accomplished by means of iontophoretic patches and the like. In certain embodiments, transdermal patches provide controlled delivery of a compound (i.e., a cyclohexenone compound described herein). In specific embodiments, the rate of absorption is slowed by using rate-controlling membranes or by trapping the compound within a polymer matrix or gel. In alternative embodiments, absorption enhancers are used to increase absorption. Absorption enhancers or carriers include absorbable pharmaceutically acceptable solvents that assist passage through the skin. For example, in one embodiment, transdermal devices are in the form of a bandage comprising a backing member, a reservoir containing the compound optionally with carriers, optionally a rate controlling barrier to deliver the compound to the skin of the host at a controlled and predetermined rate over a prolonged period of time, and means to secure the device to the skin.

Transdermal formulations described herein may be administered using a variety of devices which have been described in the art. For example, such devices include, but are not limited to, U.S. Pat. Nos. 3,598,122, 3,598,123, 3,710,795, 3,731,683, 3,742,951, 3,814,097, 3,921,636, 3,972,995, 3,993,072, 3,993,073, 3,996,934, 4,031,894, 4,060,084, 4,069,307, 4,077,407, 4,201,211, 4,230,105, 4,292,299, 4,292,303, 5,336,168, 5,665,378, 5,837,280, 5,869,090, 6,923,983, 6,929,801 and 6,946,144.

The transdermal dosage forms described herein may incorporate certain pharmaceutically acceptable excipients which are conventional in the art. In one embodiment, the transdermal formulations described herein include at least three components: (1) a formulation of a compound (i.e., a cyclohexenone compound described herein); (2) a penetration enhancer; and (3) an aqueous adjuvant. In addition, transdermal formulations can include additional components such as, but not limited to, gelling agents, creams and ointment bases, and the like. In some embodiments, the transdermal formulations further include a woven or non-woven backing material to enhance absorption and prevent the removal of the transdermal formulation from the skin. In other embodiments, the transdermal formulations described herein maintain a saturated or supersaturated state to promote diffusion into the skin.

In other embodiments, the compounds (i.e., cyclohexenone compounds described herein) are formulated for administration by inhalation. Various forms suitable for administration by inhalation include, but are not limited to, aerosols, mists or powders. Pharmaceutical compositions of a compound (i.e., a cyclohexenone compound described herein) are conveniently delivered in the form of an aerosol spray presentation from pressurized packs or a nebuliser, with the use of a suitable propellant (e.g., dichlorodifluoromethane, trichlorofluoromethane, dichlorotetrafluoroethane, carbon dioxide or other suitable gas). In specific embodiments, the dosage unit of a pressurized aerosol is determined by providing a valve to deliver a metered amount. In certain embodiments, capsules and cartridges of, such as, by way of example only, gelatins for use in an inhaler or insufflator are formulated containing a powder mix of the compound and a suitable powder base such as lactose or starch.

Intranasal formulations are known in the art and are described in, for example, U.S. Pat. Nos. 4,476,116, 5,116,817 and 6,391,452, each of which is specifically incorporated herein by reference. Formulations, which include a compound (i.e., a cyclohexenone compound described herein), which are prepared according to these and other techniques well-known in the art are prepared as solutions in saline, employing benzyl alcohol or other suitable preservatives, fluorocarbons, and/or other solubilizing or dispersing agents known in the art. See, for example, Ansel, H. C. et al., Pharmaceutical Dosage Forms and Drug Delivery Systems, Sixth Ed. (1995). Preferably these compositions and formulations are prepared with suitable nontoxic pharmaceutically acceptable ingredients. These ingredients are found in sources such as REMINGTON: THE SCIENCE AND PRACTICE OF PHARMACY, 21st edition, 2005, a standard reference in the field. The choice of suitable carriers is highly dependent upon the exact nature of the nasal dosage form desired, e.g., solutions, suspensions, ointments, or gels. Nasal dosage forms generally contain large amounts of water in addition to the active ingredient. Minor amounts of other ingredients such as pH adjusters, emulsifiers or dispersing agents, preservatives, surfactants, gelling agents, or buffering and other stabilizing and solubilizing agents may also be present. Preferably, the nasal dosage form should be isotonic with nasal secretions.

For administration by inhalation, the compounds described herein, may be in a form as an aerosol, a mist or a powder. Pharmaceutical compositions described herein are conveniently delivered in the form of an aerosol spray presentation from pressurized packs or a nebuliser, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. Capsules and cartridges of, such as, by way of example only, gelatin for use in an inhaler or insufflator may be formulated containing a powder mix of the compound described herein and a suitable powder base such as lactose or starch.

In still other embodiments, the compounds (i.e., cyclohexenone compounds described herein) are formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as, but not limited to, a mixture of fatty acid glycerides, optionally in combination with cocoa butter is first melted.

In certain embodiments, pharmaceutical compositions are formulated in any conventional manner using one or more physiologically acceptable carriers comprising excipients and auxiliaries which facilitate processing of the active compounds into preparations which can be used pharmaceutically. Proper formulation is dependent upon the route of administration chosen. Any pharmaceutically acceptable techniques, carriers, and excipients is optionally used as suitable and as understood in the art. Pharmaceutical compositions comprising a compound (i.e., a cyclohexenone compound described herein) may be manufactured in a conventional manner, such as, by way of example only, by means of conventional mixing, dissolving, granulating, dragee-making, levigating, emulsifying, encapsulating, entrapping or compression processes.

Pharmaceutical compositions include at least one pharmaceutically acceptable carrier, diluent or excipient and at least one compound (i.e., cyclohexenone compounds described herein) described herein as an active ingredient. The active ingredient is in free-acid or free-base form, or in a pharmaceutically acceptable salt form. In addition, the methods and pharmaceutical compositions described herein include the use crystalline forms (also known as polymorphs), as well as active metabolites of these compounds having the same type of activity. All tautomers of the compounds described herein are included within the scope of the compounds presented herein. The term “pharmaceutically acceptable salt” refers to those salts which retain the biological effectiveness and properties of the free bases and which are obtained by reaction with inorganic or organic acids such as hydrochloric acid, hydrobromic acid, sulfuric acid, nitric acid, phosphoric acid, methanesulfonic acid, ethanesulfonic acid, p-toluenesulfonic acid, salicylic acid, malic acid, maleic acid, succinic acid, tartaric acid, citric acid, and the like.

Additionally, the compounds described herein encompass unsolvated as well as solvated forms with pharmaceutically acceptable solvents such as water, ethanol, and the like. The solvated forms of the compounds presented herein are also considered to be disclosed herein. In addition, the pharmaceutical compositions optionally include other medicinal or pharmaceutical agents, carriers, adjuvants, such as preserving, stabilizing, wetting or emulsifying agents, solution promoters, salts for regulating the osmotic pressure, buffers, and/or other therapeutically valuable substances.

Methods for the preparation of compositions comprising the compounds described herein include formulating the compounds with one or more inert, pharmaceutically acceptable excipients or carriers to form a solid, semi-solid or liquid. Solid compositions include, but are not limited to, powders, tablets, dispersible granules, capsules, cachets, and suppositories. Liquid compositions include solutions in which a compound is dissolved, emulsions comprising a compound, or a solution containing liposomes, micelles, or nanoparticles comprising a compound as disclosed herein. Semi-solid compositions include, but are not limited to, gels, suspensions and creams. The form of the pharmaceutical compositions described herein include liquid solutions or suspensions, solid forms suitable for solution or suspension in a liquid prior to use, or as emulsions. These compositions also optionally contain minor amounts of nontoxic, auxiliary substances, such as wetting or emulsifying agents, pH buffering agents, and so forth.

In some embodiments, pharmaceutical composition comprising at least compound (i.e., cyclohexenone compounds described herein) illustratively takes the form of a liquid where the agents are present in solution, in suspension or both. Typically, when the composition is administered as a solution or suspension a first portion of the agent is present in solution and a second portion of the agent is present in particulate form, in suspension in a liquid matrix. In some embodiments, a liquid composition includes a gel formulation. In other embodiments, the liquid composition is aqueous.

In certain embodiments, pharmaceutical aqueous suspensions include one or more polymers as suspending agents. Polymers include water-soluble polymers such as cellulosic polymers, e.g., hydroxypropyl methylcellulose, and water-insoluble polymers such as cross-linked carboxyl-containing polymers. Certain pharmaceutical compositions described herein include a mucoadhesive polymer, selected from, for example, carboxymethylcellulose, carbomer (acrylic acid polymer), poly(methylmethacrylate), polyacrylamide, polycarbophil, acrylic acid/butyl acrylate copolymer, sodium alginate and dextran.

Pharmaceutical compositions also, optionally include solubilizing agents to aid in the solubility of a compound (i.e., cyclohexenone compounds described herein). The term “solubilizing agent” generally includes agents that result in formation of a micellar solution or a true solution of the agent. Certain acceptable nonionic surfactants, for example polysorbate 80, are useful as solubilizing agents, as can ophthalmically acceptable glycols, polyglycols, e.g., polyethylene glycol 400, and glycol ethers.

Furthermore, pharmaceutical compositions optionally include one or more pH adjusting agents or buffering agents, including acids such as acetic, boric, citric, lactic, phosphoric and hydrochloric acids; bases such as sodium hydroxide, sodium phosphate, sodium borate, sodium citrate, sodium acetate, sodium lactate and tris-hydroxymethylaminomethane; and buffers such as citrate/dextrose, sodium bicarbonate and ammonium chloride. Such acids, bases and buffers are included in an amount required to maintain pH of the composition in an acceptable range.

Additionally, pharmaceutical compositions optionally include one or more salts in an amount required to bring osmolality of the composition into an acceptable range. Such salts include those having sodium, potassium or ammonium cations and chloride, citrate, ascorbate, borate, phosphate, bicarbonate, sulfate, thiosulfate or bisulfite anions; suitable salts include sodium chloride, potassium chloride, sodium thiosulfate, sodium bisulfite and ammonium sulfate.

Other pharmaceutical compositions optionally include one or more preservatives to inhibit microbial activity. Suitable preservatives include mercury-containing substances such as merfen and thiomersal; stabilized chlorine dioxide; and quaternary ammonium compounds such as benzalkonium chloride, cetyltrimethylammonium bromide and cetylpyridinium chloride.

Still other pharmaceutical compositions include one or more surfactants to enhance physical stability or for other purposes. Suitable nonionic surfactants include polyoxyethylene fatty acid glycerides and vegetable oils, e.g., polyoxyethylene (60) hydrogenated castor oil; and polyoxyethylene alkylethers and alkylphenyl ethers, e.g., octoxynol 10, octoxynol 40.

Still other pharmaceutical compositions may include one or more antioxidants to enhance chemical stability where required. Suitable antioxidants include, by way of example only, ascorbic acid and sodium metabisulfite.

In certain embodiments, pharmaceutical aqueous suspension compositions are packaged in single-dose non-reclosable containers. Alternatively, multiple-dose reclosable containers are used, in which case it is typical to include a preservative in the composition.

In alternative embodiments, other delivery systems for hydrophobic pharmaceutical compounds are employed. Liposomes and emulsions are examples of delivery vehicles or carriers herein. In certain embodiments, organic solvents such as N-methylpyrrolidone are also employed. In additional embodiments, the compounds described herein are delivered using a sustained-release system, such as semipermeable matrices of solid hydrophobic polymers containing the therapeutic agent. Various sustained-release materials are useful herein. In some embodiments, sustained-release capsules release the compounds for a few hours up to over 24 hours. Depending on the chemical nature and the biological stability of the therapeutic reagent, additional strategies for protein stabilization may be employed.

In certain embodiments, the formulations described herein include one or more antioxidants, metal chelating agents, thiol containing compounds and/or other general stabilizing agents. Examples of such stabilizing agents, include, but are not limited to: (a) about 0.5% to about 2% w/v glycerol, (b) about 0.1% to about 1% w/v methionine, (c) about 0.1% to about 2% w/v monothioglycerol, (d) about 1 mM to about 10 mM EDTA, (e) about 0.01% to about 2% w/v ascorbic acid, (f) 0.003% to about 0.02% w/v polysorbate 80, (g) 0.001% to about 0.05% w/v. polysorbate 20, (h) arginine, (i) heparin, (j) dextran sulfate, (k) cyclodextrins, (1) pentosan polysulfate and other heparinoids, (m) divalent cations such as magnesium and zinc; or (n) combinations thereof.

### EXAMPLES

### Example 1: Preparation of the Exemplary Cyclohexenone Compounds

One hundred grams of mycelia from Antrodia camphorata were placed into a flask. A proper amount of water and alcohol (70-100% alcohol solution) was added into the flask and were stirred at 20-25° C. for at least 1 hour. The solution was filtered through a filter and 0.45 µm membrane and the filtrate was collected as the extract. In a instance, for example, the extracts were prepared by a solid-state fermented mycelium conditions and compositions disclosed in Lee, T-H., et al., Planta Med 2007; 73:1412-1415.

The filtrate of Antrodia camphorata was subjected to High Performance Liquid chromatography (HPLC) analysis. The separation was performed on a RP18 column, the mobile phase consisted of methanol (A) and 0.3% acetic acid (B), with the gradient conditions of 0-10 min in 95% - 20% B, 10-20 min in 20%-10% B, 20-35 min in 10%-10% B, 35-40 min in 10%-95% B, at the flow rate of 1 ml/min. The column effluent was monitored with a UV-visible detector.

The fractions collected at 21.2 to 21.4 min were collected and concentrated to yield compound 5, a product of pale yellow liquid. Compound 5 was analyzed to be 4-hydroxy-5-(11-hydroxy-3,7,11-trimethyldodeca-2,6-dienyl)-2,3-dimethoxy-6-methylcyclohex-2-enone with molecular weight of 408 (Molecular formula: C₂₄H₄₀O₅). ¹H-NMR(CDCl₃) δ (ppm)= 1.21, 1.36, 1.67, 1.71, 1.75, 1.94, 2.03, 2.07, 2.22, 2.25, 3.68, 4.05, 5.71 and 5.56. ¹³C-NMR(CDCl₃)δ(ppm): 12.31, 16.1, 16.12, 17.67, 25.67, 26.44, 26.74, 27.00, 30.10, 40.27, 43.34, 59.22, 60.59, 71.8, 120.97, 123.84, 124.30, 131.32, 134.61, 135.92, 138.05, 160.45, and 197.11.

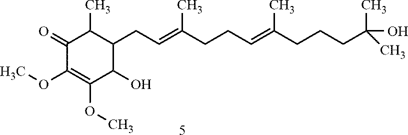

Compound 5: 4-hydroxy-5-(11-hydroxy-3,7,11-trimethyldodeca-2,6-dienyl)-2,3-dimethoxy-6-methylcyclohex-2-enone

The fractions collected at 23.7 to 24.0 min were collected and concentrated to yield compound 7, a product of pale yellow liquid. Compound 7 was analyzed to be 4-hydroxy-2,3-dimethoxy-5-(11-methoxy-3,7,11-trimethyldodeca-2,6-dienyl)-6-methylcyclohex-2-enone with molecular weight of 422 (C₂₅ H₄₂ O₅). ¹H-NMR (CDCl₃) δ (ppm) = 1.21, 1.36, 1.71, 1.75, 1.94, 2.03, 2.07, 2.22, 2.25, 3.24, 3.68, 4.05, 5.12, 5.50, and 5.61. ¹³C-NMR(CDCl₃)δ(ppm): 12.31, 16.1, 16.12, 17.67, 24.44, 26.44, 26.74, 27.00, 37.81, 39.81, 40.27, 43.34, 49.00, 59.22, 60.59, 120.97, 123.84, 124.30, 135.92, 138.05, 160.45 and 197.12.

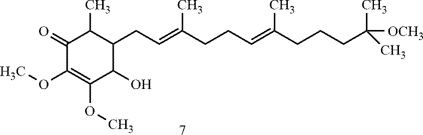

Compound 7: 4-hydroxy-2,3-dimethoxy-5-(11-methoxy-3,7,11-trimethyldodeca-2,6-dienyl)-6-methylcyclohex-2-enone

The fractions collected at 25 to 30 min were collected and concentrated to yield 4-hydroxy-2,3-dimethoxy-6-methyl-5-(3,7,1 1-trimethyldodeca-2,6,10-trienyl)cyclohex-2-enone (compound 1, also known as antroquinonol), a product of pale yellow brown liquid. The analysis of compound 1 showed the molecular formula of C₂₄H₃₈O₄, molecular weight of 390 with melting point of 48 to 52° C. NMR spectra showed that ¹H-NMR (CDCl₃) δ (ppm)=1.51, 1.67, 1.71, 1.75, 1.94, 2.03, 2.07, 2.22, 2.25, 3.68, 4.05, 5.07, and 5.14; ¹³C-NMR (CDC1₃) δ (ppm)=12.31, 16.1, 16.12, 17.67, 25.67, 26.44, 26.74, 27.00, 39.71, 39.81, 40.27, 43.34, 59.22, 60.59, 120.97, 123.84, 124.30, 131.32, 135.35, 135.92, 138.05, 160.45, and 197.12.

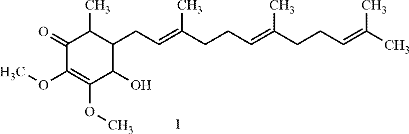

Compound 1: 4-hydroxy-2,3-dimethoxy-6-methyl-5-(3,7,11-trimethyldodeca-2,6,10-trienyl)cyclohex-2-enone

Compound 27, a metabolite of compound 1, was obtained from urine samples of rats fed with Compound 1 in the animal study. Compound 27 was determined to be 4-hydroxy-2,3-dimethoxy-6-methyl-5-(3-methyl-2-hexenoic acid)cyclohex-2-enone with molecular weight of 312 (C₁₆H₂₄ O₆). Compound 25 which was determined as 2,3-dimethoxy-5-methyl-6-((2E,6E)-3,7,11-trimethyldodeca-2,6,10-trienyl)cyclohexa-2,5-diene-1,4-dione (molecular weight of 386.52, C₂₄H₃₄O₄), was obtained from the purification process.

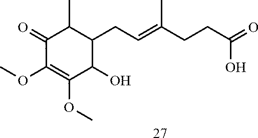

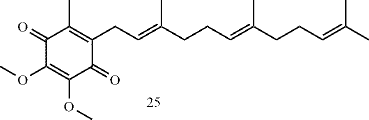

Compound 26, 4-hydroxy-2-methoxy-6-methyl-5-((2E,6E)-3,7,11-trimethyldodeca-2,6,10-trienyl)cyclohex-2-enone, was also prepared by purification process with molecular weight of 350.53 (C₂₃H₃₆O₃). Compound 28 was also prepared.

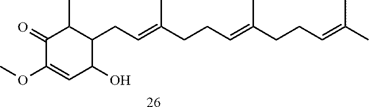

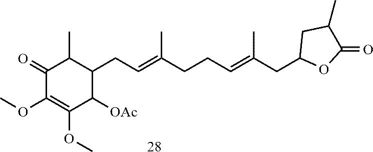

Alternatively, the exemplary compounds may be prepared from 4-hydroxy-2,3-dimethoxy-6-methylcyclohexa-2,5-dienone, or the like. See for example, see examples from U.S. Pat. No. 9,365,481 and U.S. Pat. Publication No. 2016-0237012. Similarly, other cyclohexenone compounds having the structure

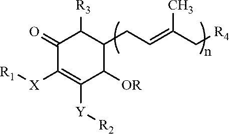

are isolated from Antrodia camphorata or prepared synthetically or semi-synthetically from the suitable starting materials. An ordinary skilled in the art would readily utilize appropriate conditions for such synthesis.

### Example 2: Anti-Viral, Anti-inflammation and Anti-Fibrosis Activities Study of Compound 1 (Antroquinonol)

An exemplary compound, Compound 1 was subject to an anti-viral, anti-inflammation and anti-fibrosis activities Study.

### Materials and Methods

Cell Culture. HepG2.2.15 cell line was cultured in MEM medium, supplemented with 10% fetal bovine serum, penicillin (100 IU/ml; Gibco, USA), and streptomycin (100 ug/ml; Gibco, USA) in 5% CO2 incubator at 37° C. This is a cell line derived from human hepatoblastoma cell line HepG2 and characterized by having stable HBV expression. Qs5 is HBV-producing rat hepatoma cell lines.

Lamivudine (3TC) and Adefovir dipivoxil (Adv) are marketed for HBV treatment as positive controls. Antroquinonol (G4) and Lamivudine (3TC) dissolved in dimethylsulfoxide (DMSO) only for stock solutions and diluted in culture medium. The final concentration of DMSO in cells is less 0.1%. Drugs treated with 2×10⁴ cells in 96-well plate for 72 hours.

The levels of HBsAg and HBeAg in HepG2.2.15 supernatants were measured using their commercial enzyme-linked immunosorbent assay (ELISA) kits following the manufacturer’s instructions.

MTT assay. 1.25 ×10⁵ cells/well were seeded onto 24- well plate. Cells were incubated with varying concentrations of G4 and 3TC (1, 5, 25, 50, 100 and 200 uM) for 72 hours. 1 mg/ml 3-(4, 5-dimethylthiazol-2-yl)-2, 5- diphenyltetrazolium- bromide (MTT) was added to each well and incubated for 2 hours at 37° C. to allow formation of the colored crystals. Medium was replaced with DMSO and plates were incubated for 15 minutes at room temperature with shaking to dissolve the crystals. The absorbance was measured by the microplate reader. The optical density was measured at 490 nm.

Southern blot analysis of viral DNA. HBV core particle-associated DNAs were resolved on a 1.2% native agarose gel and detected by Southern blot analysis, using a specific HBV DNA probe. Each lane on the Southern blot gel was loaded with the total amount of core particle-associated viral DNA extracted from each treatment dish, which was seeded with equal cell density one night before transfection.

### Results

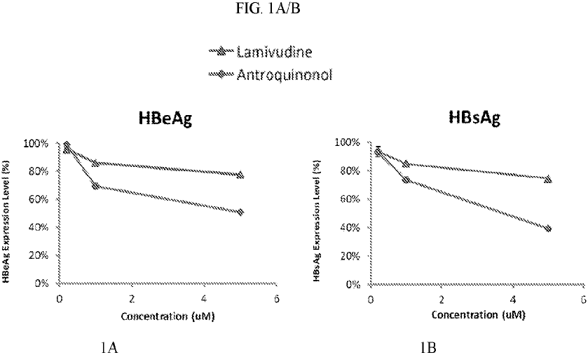

To examine the effect of Antroquinonol on HBV protein expression, including surface antigen (HBsAg) and e antigen (HBeAg), ELISA assay was used to quantify HBV replication. The study results have shown that Compound 1 (i.e., Antroquinonol) reduces HBeAg (see FIG. 1A) and HBsAg (see FIG. 1B) expression levels. Compound 1 in comparison with Lamivudine, has shown reduction in the expression levels of the two major HBV biomarkers, HBeAg and HBsAg by 50% and 40% respectively. Thus, in short summary, the exemplary compound 1 shows significantly suppressed effects not only on HBeAg but also HBsAg at the different doses.

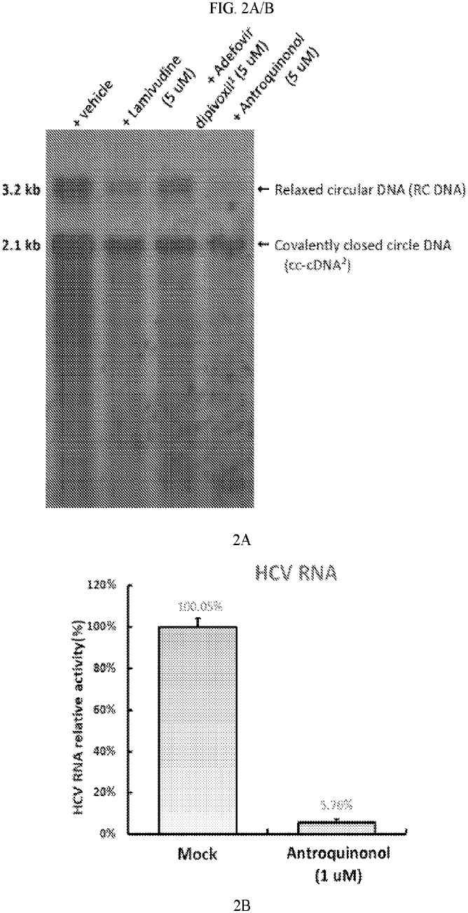

To evaluate the effect of the exemplary Compound 1 on viral replication, the intracellular HBV core particle-associated DNA was isolated and analyzed. The exemplary compound 1, Antroquinonol, shows significantly suppressed HBV replicative intermediates (relaxed-circular, linear and single-stranded DNA) by Southern blotting. As shown in FIG. 2A/B, Compound 1 (Antroquinonol) reduces HBV DNA expression levels (2A), and reduces HCV RNA activity (2B). In particular, Compound 1, in comparison with Lamivudine and Adefovir dipivoxil, has shown more apparent results in reducing the expression levels of the HBV DNA. Compound 1 also shows a drastic reduction in HCV RNA activity by 95%.

Suppression of HBV production by Antroquinonol might be a result of its cytotoxicity, and this possibility was examined by using MTT assay. No apparent cytotoxicity was detected up to exposure to 5 uM of Antroquinonol, suggesting that the suppression of supernatant viral protein and DNA levels by Antroquinonolwas not caused by its cytotoxicity. Of note, over 25 uM of Antroquinonolhad cytotoxicity effects on HepG2.2.15 cells.

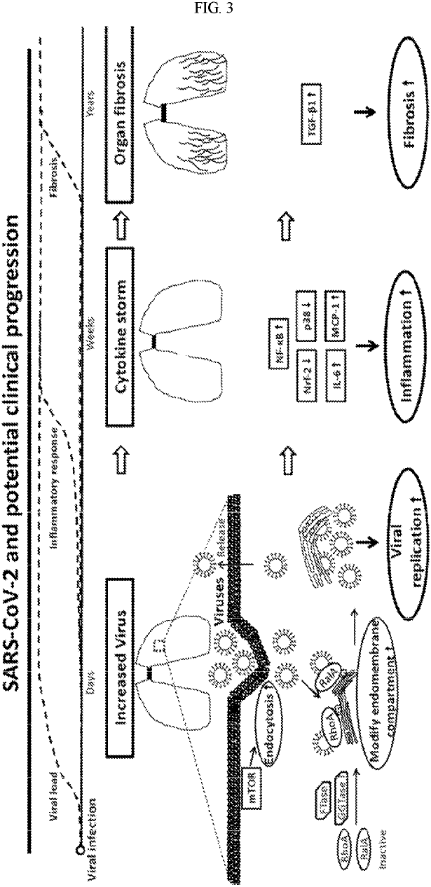

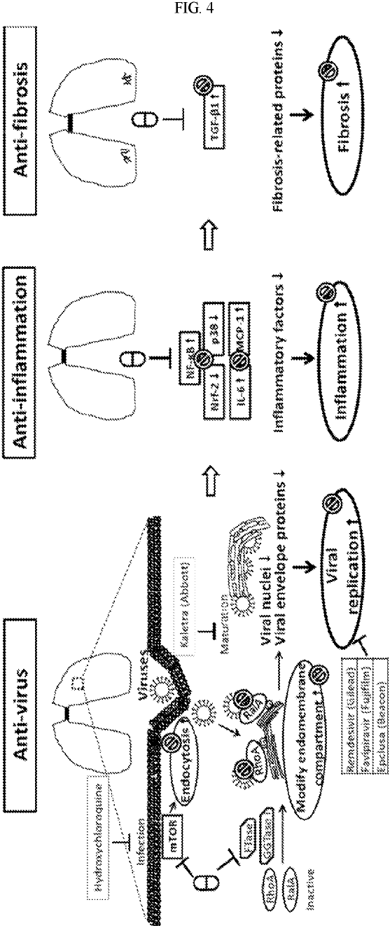

To treat, reduce symptoms of, or prevent the course of coronavirus infection (such as SARS-CoV-2) in a subject, it is understood that the multiple approaches besides anti-viral activities are needed as illustrated in FIG. 3. After the viral infection, the virus will replicate and the number of virus are increased, the inflammation occurs causing cytokine storm, resulting lump fibrosis. Thus, a compound such as the exemplary Compound 1 with the ability of anti-virus, anti-inflammation and even anti-fibrosis is a suitable medicine against coronavirus infection, as illustrated in FIG. 4. For example, it is shown that Compound 1 inhibits the function of mTOR and inhibits endocytosis.

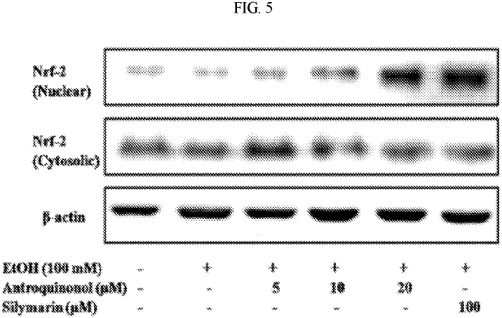

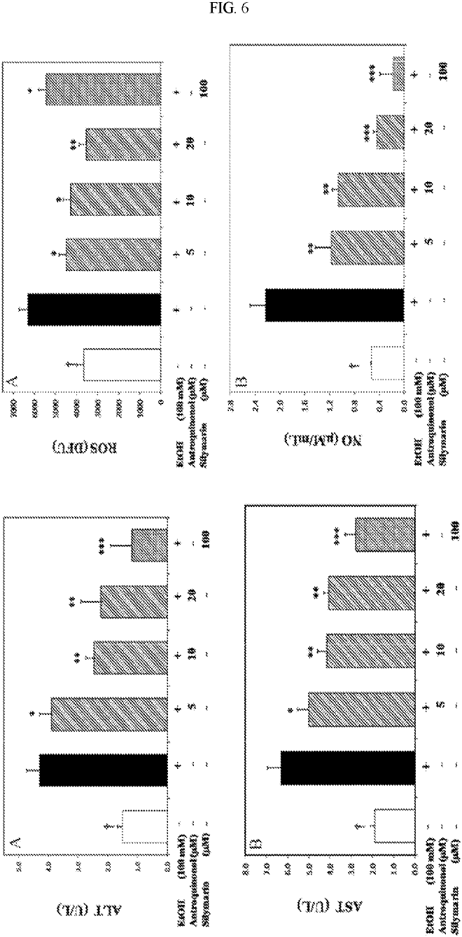

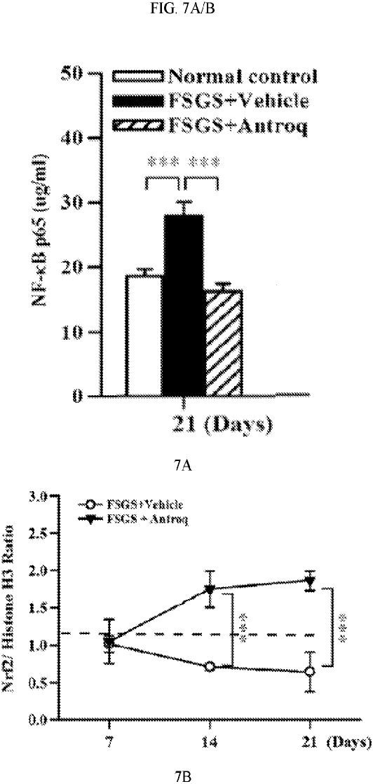

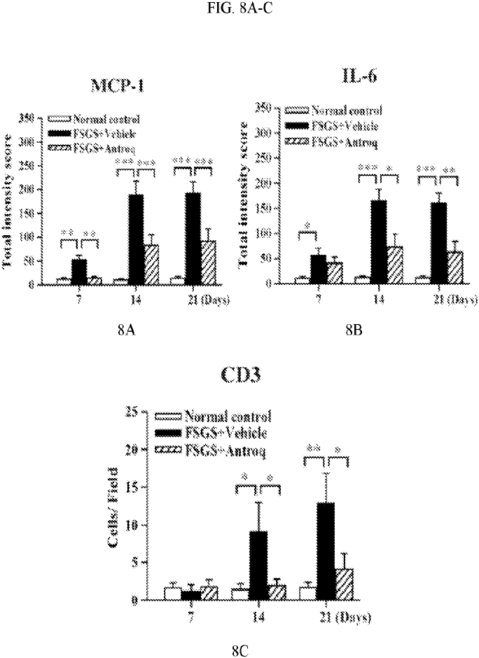

In particular, the study results (see FIG. 5) provide that Compound 1 in comparison with silymarin, shows effective increase in Nrf-2 nuclear translocation at lower concentrations of administration. FIG. 6 provides that Compound 1 significantly reduced ethanol-induced elevation of ALT and AST and suppressed oxidative stress. Other anti-inflammation results from Compound 1 include the suppression in NF-kB expression by 36%, as well as 2 times enhancement in nuclear Nrf-2 expression as shown in FIG. 7. Compound 1 also shows to effectively suppress MCP-1, IL-6, and CD3 expression by around 50%, 57%, and 66% respectively in FIG. 8. All the study results above suggest its effectiveness in anti-inflammation activity by Compound 1.

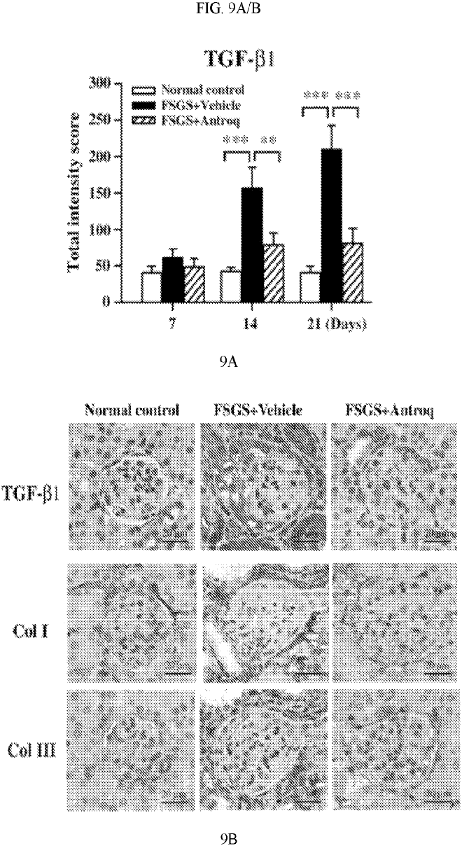

It was also found that the exemplary Compound 1 provides anti-fibrotic activity. FIG. 9A illustrates that exemplary Compound 1 effectively suppress the expression of TGF- β1 by around 64%. Compound 1 also shows anti-fibrosis property as shown in FIG. 9B in study utilizing the fibrosis related proteins (Col 1 and Col III). Thus, the data clearly indicated that Compound 1 ablates the viral activity, the protein expression of inflammatory effectors, and the TGFβ1 signal-mediated fibrosis.

### Example 3: Impact Study of Exemplary Compound 1 on COVID-19 Progression

The study aims for evaluating the impact of exemplary Compound 1 on COVID-19 (i.e., SARS-CoV-2) progression by anti-SARS-CoV-2 (Specific Aim 1), anti-SARS-CoV-2-induced cytokine storm, and anti-SARS-CoV-2-induced fibrosis (Specific Aim 2). The overall objective is to confirm if the exemplary Compound 1 (i.e., Antroquinonol) provide a potential triple action for COVID-19 treatment and offer a new therapeutic regimen for patients with SARS-CoV-2.

### Specific Aim 1. Investigate the Functional Effects of Antroquinonol on Anti-SARS-CoV-2

The yield reduction assay was used to determine the inhibition rate of Antroquinonol (EC₅₀) against SARS-CoV-2. Briefly, Vero E6 cells seeding into 24-well culture plates in DMEM with 2% FBS and were treated with Compound 1 (i.e., Antroquinonol, 10 or 20 µM) for 1 h. The plates without any treatments in DMSO were used as control. Then, the resulted cells were infected with SARS-CoV-2 (multiplicity of infection, MOI = 0.1) for 1 h. After removal of Antroquinonol and virus, the cells were washed once with PBS and overlaid with overlay medium containing different concentrations of Antroquinonol for 24 h. The cell culture media were collected for viral plaque assay to determine the number of plaque-forming units. Vero E6 cells were seeded into 24-well culture plates in DMEM with 10% FBS and antibiotics 1 day before infection. The cell culture media was adding to the cell monolayer and incubated for 1 h at 37° C. Subsequently, cell culture media were removed, and the cell monolayer was washed once with PBS before covering with media containing 1% methylcellulose for 5 days. The cells were fixed with 10% formaldehyde 1 hour. After removal of the overlay media, the cells were stained with 0.5% crystal violet, and the plaques were counted. The cells were collected for protein and RNA extraction by AMRESCO’s RIPA cell lysis buffer and NucleoSpin RNA Kit (Macherey-Nagel), respectively. Then, the expression levels of the nucleocapsid protein and the E gene were detected by Western blot (Antibody cat no. 40143-R019) and quantitative real-time PCR (qRT-PCR), respectively. Moreover, isolated RNAs were used for Specific Aim 2. Also, cytotoxicity (i.e. IC₅₀) of Antroquinonol on Vero E6 cells will be measured by an acid phosphatase assay. Here, the Remdesivir (1 µM) treatment will be used as a control. All results will be showed as the mean ± s.d. from at least three independent experiments.

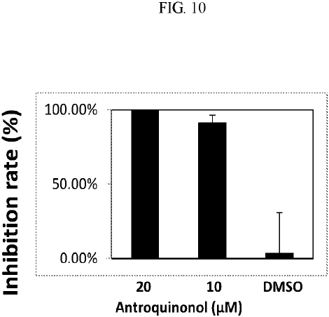

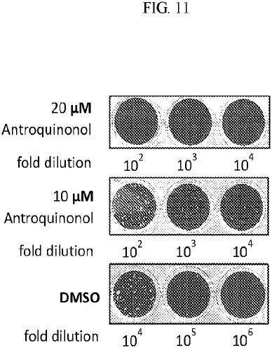

The study results show that Compound 1 in both 20 and 10 µM reduced SARS-CoV-2 concentration substantially (99.93% for 20 µM and 91.20 % for 10 µM). See FIG. 10. FIG. 11 also provides the cell culture results showing the cell culture plates from the treatment of Antroquinonol and control (DMSO plates).

### Specific Aim 2. Explore the Effects of Antroquinonol on SARS-CoV-2-Induced Cytokine Storm and SARS-CoV-2-Induced Fibrosis

Current studies indicated that several cytokines/chemokines were significantly correlated to COVID-19 disease. For example, plasma IP-10 (also known as CXCL10) is highly associated with disease severity and predicts the progression of COVID-19. IL-6 could also function as a predictor of progression to severe COVID-19, suggesting targeting cytokines as a therapeutic option in patients with COVID-19. Regarding the long-term effects of COVID-19, TGF-β-mediated collagen deposition could function as an important contributor in the irreversible pulmonary fibrosis.

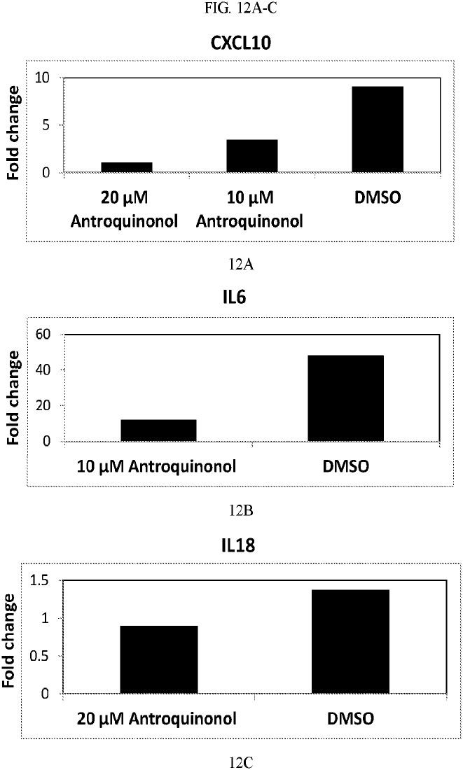

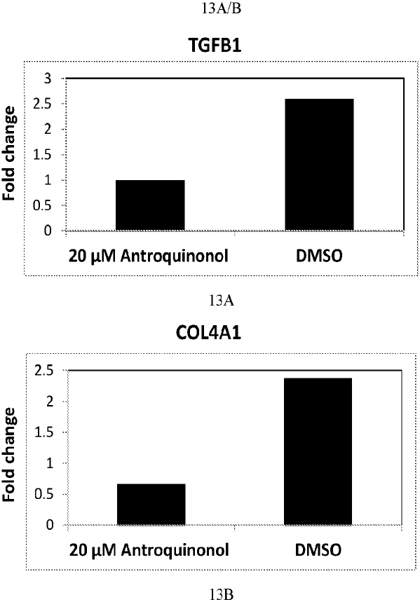

To reveal the effects of Antroquinonol on SARS-CoV-2-induced cytokine storm and SARS-CoV-2-induced fibrosis, RNAs described in Specific Aim 1 were used to detect the gene expression of cytokines/chemokines (such as CXCL10, IL6, and IL18, see FIGS. 12A-C), pro-fibrotic growth factor (such as TGFB1, see FIG. 13A) and collagen (such as COL1A1, COL3A1, and COL4A1). Briefly, 5.4 µg RNA was reversely transcribed into cDNA by M-MLV Reverse Transcriptase Kit. Real-time PCR analysis was set up with SYBR™ Green Master Mix Kit and carried out in QuantStudio™ 5 Real-Time PCR System. The relative level of target mRNA was determined by normalizing actin rRNA.

### Study Results

FIGS. 12A-C provides the gene expression levels of CXCL10 (12A), IL6 (12B), and IL18 (12C), respectively. With 20 µM of Antroquinonol, there was a 1.01-fold change in CXCL10 expression, while a 3.40-fold change and 9.04 fold change were observed with 10 µM of Antroquinonol and DMSO, respectively. With 10 µM of Antroquinonol, there was a 11.88-fold change in IL6 expression, while a 47.81-fold change was observed with DMSO. With 20 µM of Antroquinonol, there was a 0.89-fold change in IL18 expression, while a 1.36-fold change was observed with DMSO.

FIGS. 13A-B provides the gene expression levels of TGFB1(13A), and COL4A1 (13B), respectively. With 20 µM of Antroquinonol, there was a 0.99-fold change in TGFB1 expression, while a 2.59-fold change was observed with DMSO. With 20 µM of Antroquinonol, there was a 0.65-fold change in COL4A1 expression, while a 2.37-fold change was observed with DMSO.

Thus, it is clearly shown that the exemplary compound, Antroquinonol provide superior effects on SARS-CoV-2-induced cytokine storm and SARS-CoV-2-induced fibrosis.

### Example 4: A Clinical Trial of Phase 2 Study to Evaluate the Safety and Efficacy of Compound 1 in Hospitalized Patients with Mild to Moderate Pneumonia Due to COVID 19

The primary objectives of this study are:
To evaluate the efficacy of antroquinonol treatment of mild to moderate pneumonia due to COVID-19, as measured by:
   time to clinical improvement
   progression of disease.
To evaluate the safety of antroquinonol treatment in patients with mild to moderate pneumonia due to COVID-19.
The secondary objectives are:
To further evaluate the efficacy of antroquinonol compared with placebo in this patient population as measured by:
   duration of hospitalization
   virological clearance
   vital status (death)
To assess the pharmacokinetics (PK) plasma concentrations of antroquinonol in this patient population.
To assess the safety of antroquinonol in this patient population.

### Study Design

This is a Phase 2 clinical trial to evaluate the safety and efficacy of antroquinonol in hospitalized patients with mild to moderate pneumonia due to COVID-19.

The main characteristics of hospitalized patients to be included in this study are: adult patients with a fever onset within 5 days prior to screening along with respiratory rate > 24/minute. Symptoms of mild to moderate pneumonia due to COVID 19 (confirmed findings on chest × ray or computerized scan [CT] scan) must have been present. The planned treatment duration is 10 days of administration of antroquinonol or placebo in combination with standard of care (SoC) therapy per local SoC policies.

A total of 166 patients are planned to be enrolled and randomized in a 1:1 ratio of antroquinonol to placebo.

As antroquinonol has shown antiviral and anti-inflammatory activity in pre-clinical studies, it is being planned to use for treatment in patients with COVID-19 infection. Therefore, an initial sentinel cohort of patients is planned to be treated to assess the safety of antroquinonol. This sentinel cohort will comprise the first 20 patients (10 patients assigned to antroquinonol and 10 patients to placebo). Enrollment will pause once the first 20 patients have started treatment.

The Data Monitoring Committee (DMC) will assess the safety and tolerability of antroquinonol in this sentinel cohort once the first 20 patients have completed at least 10 days of therapy. The DMC may unblind the data for this assessment. Once the 20 patients in the sentinel cohort have been treated for at least 10 days, and the study has been assessed by the DMC as safe to continue, enrollment will resume.

All patients enrolled in the study (including the sentinel cohort) will be included in the primary analysis of efficacy and safety of study treatment. The DMC will review safety and assess risk/benefit profile on an ongoing basis. The primary efficacy analysis will be conducted once all patients have achieved clinical improvement, or have been followed for 28 days from the start of therapy.

### Number of Patients

A total of 166 patients are planned to be enrolled in the study (83 patients in the antroquinonol group and 83 patients in the placebo group). This enrollment level ensures approximately 135 improvement events. Enrollment will be based on the following assumptions:

The randomized allocation ratio is 1:1 between the antroquinonol group and the placebo group;
Clinical improvement is defined as change in median from 7 days to 4 days.
Each patient will be followed for up to 28 days
Power of 90%
Two sided alpha of 0.05.

### Diagnosis and Main Criteria for Inclusion and Exclusion

Patients must satisfy all of the following inclusion criteria at the Screening visit unless otherwise stated:
1. Willing and able to provide informed consent.
2. Male or female patients between ≥18 and ≤80 years of age.
3. Hospitalized with fever (defined as oral temperature ≥ 38.6° C.) and respiratory rate > 24/minute. Fever (armpit ≥ 36.6° C., or mouth ≥ 37.2° C., or anal or ear ≥ 37.8° C...Gilead) Note: Hospitalized patients can also include patients admitted to centers conditioned as hospitals to treat COVID-19 patients.
4. Chest x-ray or CT scan consistent with pneumonia. Status: Unilateral and bilateral pneumonia (infiltrates/interstitial)
5. Fever onset within 5 days prior to screening.
6. SARS CoV 2 infection confirmed by a PCR test of nasopharyngeal sample (not serology testing).
7. Male patients and female patients of childbearing potential must agree to use protocol-specified methods of contraception.
8. Females patients of childbearing potential must have a negative pregnancy test at Screening and pre-treatment on Day 1.
9. Male patients must agree not to donate sperm from the first dose through 90 days after the last dose of study drug.
10. Patient is, in the opinion of the investigator, willing and able to comply with the study drug regimen and all other study requirements.
11. Hospitalized < 48 hours and Randomization within 48 hours of meeting inclusion criteria

Patients will be excluded from the study if they satisfy any of the following exclusion criteria at the Screening visit unless otherwise stated:
1. Female patient is pregnant or breastfeeding.
2. Any patient’s concomitant life threatening condition, including but not limited to: requiring mechanical ventilation, acute respiratory distress syndrome (ARDS), shock, or cardiac failure.
   Patients need invasive mechanical ventilation; or other organ failure need ICU monitoring Is it acceptable for oxygen therapy (O2 inhalation) by mask?
3. Evidence of lobar or sub lobar consolidation on chest × ray.
4. Blood oxygen saturation (SpO2) < 90% in room air, or partial pressure of arterial oxygen (PaO2)/percentage of inspired oxygen (FiO2) < 200 mmHg, severe dyspnea or requiring positive pressure ventilation with or without intubation
5. Abuse of drugs or alcohol that could interfere with adherence to study requirements, as judged by the investigator.
6. Treatment with other drugs thought to possibly have activity vs COVID 19 within 7 days prior to enrollment.
7. Use of other investigational drugs within 30 days of dosing, or plans to enroll in another clinical trial of an investigational agent while participating in the present study.
8. Clinically significant abnormal ECG at Screening, as determined by the investigator.
9. Patient requires frequent or prolonged use of systemic corticosteroids or other immunosuppressive drugs (e.g., for organ transplantation or autoimmune conditions).
10. Abnormal laboratory values at Screening:
   a. Estimated glomerular filtration rate (GFR) <50 mL/min.
   b. Alanine aminotransferase (ALT) or aspartate aminotransferase (AST) >5 × upper limit of normal (ULN), or ALT/AST >3 × ULN plus total bilirubin >2 × ULN.
   c. Platelet count < 100 × 109/L.
   d. Total bilirubin >1.5 × ULN, unless the patient has known Gilbert’s syndrome.
   e. Hemoglobin < 9 g/dL for females or <11 g/dL for males.
   f. Total white blood cell (WBC) count < 3,500/mm3 or absolute neutrophil count (ANC) < 1,500/mm3.
11. Treatment with any anti viral drug(s) or with any drugs known to be strong inducers or inhibitors of CYP2C19, CYP3A4, CYP2C8 and CYP2E1 within 14 days prior to the start of study treatment.
12. Any other clinically significant medical condition or laboratory abnormality that, in the opinion of the investigator, would jeopardize the safety of the patient or potentially impact patient compliance or the safety/efficacy observations in the study.
13. Viral pneumonia with other viruses besides 2019-nCoV
14. Patients can’t take drugs orally
15. Patients intubated or requiring imminent intubation at the time of randomization
16. Severe cognitive and mental disorders

### Test Products, Dose, and Mode of Administration

Antroquinonol (100 mg capsule) in a dose of 200 mg (2 capsules) administered twice daily (BID) orally for 10 days.

Reference therapy, dose, dose form, and mode of administration: Placebo (capsule) administered orally BID for 10 days.

Duration of patient participation in study:
Total study duration is planned to be up to 28 ±2 days.
The screening period is planned to be up to 2 days. The planned treatment duration is of 10 days. Follow up safety assessments will be performed at Days 14 and 28 (±2 days).

Study populations: Full Analysis Set (FAS): All randomized patients who have received at least 1 dose of the study drug. Patients will be analyzed according to the treatment to which they were randomized.

Per Protocol Set (PPS): All patients from FAS who have no important study protocol deviations during the study. Patients with any important protocol deviations shall be excluded from PPS prior to database lock.

Safety Set (SS): All patients who have received at least 1 dose of the study drug. Patients will be analyzed according to the study treatment they actually received.

Pharmacokinetic Set (PKS): All patients who have received at least 1 dose of the study drug and have at least 1 evaluable plasma concentration without important protocol deviations or events thought to significantly affect the PK.

### Endpoints

The primary efficacy endpoints are: Time to clinical improvement. [Time Frame: within 28 days from the start of medication]

Clinical improvement is defined as the time (days) from start of study treatment until normalization of fever ≤ 37.2° C. oral, respiratory rate of ≤ 24/minute on room air, and blood oxygen saturation (SpO2) of > 94% on room air. (individual or entire)

Resolution of hypoxia (defined as SpO2 ≥ 93% on room air OR Pa02/Fi02 ≥ 300 mmHg)
Rate of disease remission [Time Frame: on days 14 and 28 from the start of medication]
Progression of disease

Progression of disease is defined as requirement of positive pressure ventilation with or without intubation, or requirement of ICU care. For the subset of patients who remain hospitalized and have arterial blood gas (ABG) test performed as part of SoC, PaO2/FiO2 < 200 mmHg will also be used as a measure of progression of disease. Rate of invasive mechanical ventilation when respiratory failure occurs [Time Frame: by 10 days].

The secondary efficacy endpoints are:
Duration of hospitalization (days).
Virological clearance from nasopharyngeal or respiratory samples
   Time to virological clearance, measured as study days from start of treatment to first negative SARS-CoV-2 PCR test
   Rate of change in viral load will be evaluated depending on availability of quantitative assays. (Real-time RT-PCR test)
   Vital status (death) will be collected up to Day 14 and Day 28
   Lung imaging improvement time [Time Frame: within 10 days after taking medicine]
The safety endpoints include the following variables:
Adverse events (AEs).
Chest imaging (x-ray or CT scan) findings.
Vital signs: blood pressure, pulse rate.
Physical examination: general appearance, HEENT, lymphatic, cardiovascular, respiratory, gastrointestinal, musculoskeletal, neurological, dermatological.
12 lead electrocardiogram (ECG).
Standard safety laboratory tests (hematology, chemistry and urinalysis).

### Pharmacokinetic Evaluation

The PK parameters to be assessed from plasma samples are:
Trough (pre dose) plasma concentration (Ctrough)
Maximum plasma concentration (Cmax).

### Statistical Methods

### General Principles

Continuous variables will be summarized by the standard descriptive statistics: number of patients (n), mean, standard deviation (SD), median, minimum (min) and maximum (max). Frequency of patients or events and percentages will be summarized in categorical variables. Results will be considered statistically significant at one-sided alpha of 0.025, and considered to indicate promising trend at one-sided alpha of 0.2.

### Efficacy Analysis

### Primary Efficacy Analysis

The hazard ratio (HR) and its 95% confidence interval (CI) for time to clinical recovery will be estimated by Cox proportional hazard model, with patients censored at the time of death, at the time they are provided any non-study anti-viral therapy, or on Day 28 if they have not yet recovered. Median time to clinical improvement will be estimated by Kaplan-Meier (KM) method, and the KM curve will be provided. The p-value for comparison between groups will be obtained based on log-rank test.

For progression of diseases, the proportion of patients requiring positive pressure ventilation and requirement of ICU care in both groups and difference between groups and 95% CI will be calculated using logistic regression. The P-value will be based on Chi-square test. For the subset of patients who have ABG data collected as part of SoC, PaO₂/FiO₂ will also be evaluated.

### Secondary Efficacy Analysis

Duration of hospitalization, virological clearance (time to virological clearance, rate of change in viral load), and vital status will be analyzed using similar statistical methods as the primary efficacy endpoints.

### Safety Analysis:

Adverse events will be coded according to the Medical Dictionary for Regulatory Activities (MedDRA).

The number and percentage of patients with treatment emergent AEs (TEAEs), serious AEs (SAEs), TEAEs related to study treatment, SAEs related to study treatment TEAEs leading to treatment discontinuation, TEAEs leading to study discontinuation, and TEAEs leading to death will be summarized by system organ class (SOC), preferred term (PT) and treatment groups. In addition, the severity of TEAEs and relationship to study treatment will be summarized by SOC, PT, and treatment groups.

The following Standardized MedDRA Queries (SMQ) identify AEs of special interest (AESIs), and will be reported:
Respiratory failure
Opportunistic infections

Test values and changes from baseline will be summarized descriptively for specific laboratory test results, vital signs, SpO2, physical examinations, and ECG findings. Where applicable, shift tabulations by treatment groups will be presented. Pharmacokinetic Analysis:

Descriptive statistics will be provided for antroquinonol plasma concentration and/or PK parameters.

TABLE 1

Schedule of Assessments

| Study Period | Screening | Hospitalization | | Follow-up | |
|---|---|---|---|---|---|
| Visit name | | (Baseline) Treatment | Hospital Discharge and/or EOT | | |
| Study Day(s) | -2 to 0 | 1 | 2 to 10^{a} | 14±2^{a} | 28 ± 2 or EOS^{a} |
| Informed consent | X | | | | |
| Randomization^{b} | | X | | | |
| Study Day(s) | -2 to 0 | 1 | 2 to 10^{a} | 14±2^{a} | 28 ± 2 or EOS^{a} |
| Inclusion/exclusion criteria | X | | | | |
| Demographics, medical history | X | | | | |
| Physical exam^{c} | X | X | X | X | X |
| Height, body weight | X | | | | |
| Vital signs (temperature, respiratory rate, blood pressure, and pulse)^{c} | X | X | X | X | X |
| Chest x-ray or CT scan^{d} | X | | X | | |
| Urine pregnancy test^{e} | X | | | | X |
| Study medication or placebo | | X | X | | |
| COVID-19 PCR test | X | X | X | X | X |
| Ventilator support status | X | X | X | | |
| Assessment of clinical worsening | | X | X | | |
| SpO₂ | X | X | X | X | X |
| Arterial blood gas evaluation (PaO₂/FiO₂) | X | X | X | | |
| Adverse events | | X | X | X | X |
| Study Day(s) | -2 to 0 | 1 | 2 to 10^{a} | 14±2^{a} | 28 ± 2 or EOS^{a} |
| Laboratory assessments | | X | X | | |
| Electrocardiogram | | X | | | X |
| Prior and concomitant medications | X | X | X | X | X |
| PK parameters | | X | X | | |

Abbreviations: AE = adverse event; Cₘₐₓ= maximum plasma concentration; C_{trough}= trough (pre-dose) plasma concentration; CT=computerized scan; DMC=data monitoring committee; ECG = electrocardiogram; EOS=end of study; EOT=end of treatment; FiO₂=percentage of inspired oxygen; HEENT=head, eyes, ear, nose and throat; ICU = intensive care unit; PaO₂= partial pressure of arterial oxygen; PCR = polymerase chain reaction; PD = pharmacodynamic; PK = pharmacokinetic; SpO₂ = blood oxygen saturation/pulse oximetry. Footnotes:
a Patients could be discharged after start of treatment anytime during Days 2 to 10 after achieving clinical recovery (defined as the time [days] from start of study treatment until normalization of fever and respiratory rate and oxygenation). Patients will then be requested to take treatments at home (as prescribed) and to be followed-up via telephone call on Days 14 and 28 for assessment of symptoms. Discharged patients are required to visit the hospital/site to complete the assessments for Day 10/EOT visit.
b Initial cohort of 20 patients to be enrolled to assess safety and tolerability. Once the DMC confirms no safety concerns, study will resume enrolling remaining patients.
c Body temperature (oral, forehead, axillary, tympanic) of ≥38.6° C. within 5 days prior to screening and respiratory rate >24/minute. A complete physical examination (general appearance, HEENT, lymphatic, cardiovascular, respiratory, gastrointestinal, musculoskeletal, neurological, and dermatological systems) will be performed at screening. COVID-19 symptom-targeted physical examination will be performed during hospitalization. Vital signs (respiratory rate, blood pressure and pulse rate) to be assessed daily during hospitalization. Height and body weight will be measured only at screening.
d Chest x-ray or CT scan should show findings consistent with COVID-19 pneumonia and will be performed at screening and hospital discharge.
e Urine pregnancy test to be performed in female patients of child-bearing potential and in local (site) lab.
f Efficacy parameter assessments: [1] PCR testing for COVID-19 (until negative results) to performed at a local laboratory at Screening and subsequent visits. This test could be performed at a central laboratory for discharged patients for their subsequent tests; [2] Assessment of clinical worsening status could be assessed if the patient requires prolonged hospitalization or progression of disease (defined as requirement of positive pressure ventilation with or without intubation, or requirement of ICU care. For the subset of patients who remain hospitalized and have arterial blood gas (ABG) test performed as part of SoC, PaO₂/FiO₂ will also be used as a measure of progression of disease); [3] Daily SpO₂ monitoring on Day 1: average of 3 consecutive readings in a 5-minute period; [4] Arterial blood gas evaluation of PaO₂/FiO₂ at screening and during hospitalization (until discharge).
g Safety parameter assessments: [1] AEs will be assessed from the start of treatment (during hospitalization and post discharge at home) until EOS; [5] Standard safety laboratory tests will include all parameters of hematology, chemistry and urinalysis and will performed pre-dose on Days 1, 5, and 10; [6] 12-lead ECGs to be performed at screening while the patient is in supine position; [7] prior and concomitant medications to be recorded from screening until EOS.
h Pharmacokinetic parameters include Ctrough, and Cmax. Blood samples for these parameters will be assessed on Day 5 and Day 10 (if patients are still hospitalized), pre-dose and 2-hour post-dose.

### Example 5: Oral Formulation

To prepare a pharmaceutical composition for oral delivery, equal weight amount of an exemplary Compound 1 was mixed with equal weight amount of corn oil (e.g., 25 mg, 50 mg, 100 mg, 200 mg). The mixture was incorporated into an oral dosage unit in a capsule, which is suitable for oral administration.

In some instances, 100 mg of a compound described herein is mixed with 750 mg of starch. The mixture is incorporated into an oral dosage unit for, such as a hard gelatin capsule, which is suitable for oral administration.

### Example 6: Sublingual (Hard Lozenge) Formulation

To prepare a pharmaceutical composition for buccal delivery, such as a hard lozenge, mix one part of a compound described herein, with 4 to 5 parts of powdered sugar mixed, with suitable amount of light corn syrup, distilled water, and mint extract. The mixture is gently blended and poured into a mold to form a lozenge suitable for buccal administration.

### Example 7: Inhalation Composition

To prepare a pharmaceutical composition for inhalation delivery, 20 mg of a compound described herein is mixed with 50 mg of anhydrous citric acid and 100 mL of 0.9% sodium chloride solution. The mixture is incorporated into an inhalation delivery unit, such as a nebulizer, which is suitable for inhalation administration.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for treating or reducing symptoms of or preventing an RNA virus induced disease in a subject comprising administering to said subject a therapeutically effective amount of a cyclohexenone compound having the structure: 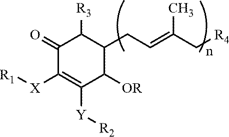
wherein each of X and Y independently is oxygen, NR₅ or sulfur;
R is a hydrogen or C(=O)C₁-C₈alkyl;
each of R₁, R₂ and R₃ independently is a hydrogen, optionally substituted methyl or (CH₂)ₘ——CH₃;
R₄ is NR₅R₆, OR₅, OC(═O)R₇, C(═O)OR₅, C(═O)R₅, C(═O)NR₅R₆, halogen, 5 or 6-membered lactone, C₁-C₈alkyl, C₂-C₈alkenyl, C₂-C₈alkynyl, aryl, glucosyl,
wherein the 5 or 6-membered lactone, C₁-C₈alkyl, C₂-C₈alkenyl, C₂-C₈alkynyl, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈
alkynyl, C₃-C₈ cycloalkyl, and C₁-C₈ haloalkyl;
each of R₅ and R₆ is independently a hydrogen or C₁-C₈alkyl;
R₇ is a C₁-C₈alkyl, OR₅ or NR₅R₆;
m = 1-12; and
n=1-12; or a pharmaceutically acceptable salt, metabolite, solvate or prodrug thereof.

2. The method of claim 1, wherein the RNA virus is a coronavirus.

3. The method of claim 1, wherein the RNA virus induced disease is caused or induced by a coronaviridae infection.

4. The method of claim 1, wherein the cyclohexenone compound reduces RNA virus concentration or prevents RNA virus infection.

5. The method of claim 1, wherein said RNA virus induced disease is a coronavirus induced pneumonia, or a SARS-CoV-2 induced pneumonia.

6. The method of claim 5, wherein said RNA virus induced disease is an RNA virus induced pneumonia.

7. The method of claim 3, where said coronaviridae infection is caused by or associated with alpha coronaviruses 229E (HCoV-229E), NL63 (HCoV-NL63, New Haven coronavirus), beta coronaviruses OC43 (HCoV-OC43), HKU1, MERS-CoV (the coronavirus responsible for Middle East Respiratory Syndrome), SARS-CoV, severe acute respiratory syndrome coronavirus 2 (SARS-CoV-2), or SARS-CoV-2 (2019-nCoV).

8. The method of claim 7, wherein said coronaviridae infection is caused by or associated with severe acute respiratory syndrome coronavirus 2 (SARS-CoV-2).

9. The method of claim 1, wherein said cyclohexenone compound, or a pharmaceutically acceptable salt, metabolite, solvate or prodrug thereof, is administered orally, parenterally or intravenously.

10. The method of claim 1, wherein said cyclohexenone compound, or a pharmaceutically acceptable salt, metabolite, solvate or prodrug thereof, is administered by injection.

11. The method of claim 1, wherein said subject is human.

12. The method of claim 1, wherein R is a hydrogen, C(═O)C₃H₈, C(═O)C₂H₅, or C(═O)CH₃.

13. The method of claim 1, wherein each of R₁, R₂ and R₃ independently is a hydrogen, methyl, ethyl, propyl, butyl, pentyl, hexyl, heptyl, or octyl.

14. The method of claims 13, wherein R₁ is a hydrogen or methyl.

15. The method of claims 13, wherein R₂ is a hydrogen or methyl.

16. The method of claim 1, wherein R₄ is halogen, NH₂, NHCH₃, N(CH₃)₂, OCH₃, OC₂H₅, C(═O)CH₃, C(═O)C₂H₅, C(═O)OCH₃, C(═O)OC₂H₅, C(═O)NHCH₃, C(═O)NHC₂H₅, C(═O)NH₂, OC(═O)CH₃, OC(═O)C₂H₅, OC(═O)OCH₃, OC(═O)OC₂H₅, OC(═O)NHCH₃, OC(═O)NHC₂H₅, or OC(═O)NH₂.

17. The method of claim 1, wherein R₄ is C₂H₅C(CH₃)₂OH, C₂H₅C(CH₃)₂OCH₃, CH₂COOH, C₂H₅COOH, CH₂OH, C₂H₅OH, CH₂Ph, C₂H₅Ph, CH₂CH═C(CH₃)(CHO), CH₂CH═C(CH₃)(C(═O)CH₃), 5 or 6-membered lactone, aryl, or glucosyl, wherein the 5 or 6-membered lactone, aryl, and glucosyl are optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl.

18. The method of claim 1, wherein R₄ is C₁-C₈alkyl optionally substituted with one or more substituents selected from NR₅R₆, OR₅, OC(═O)R₇, C(═O)OR₅, C(═O)R₅, C(═O)NR₅R₆, C₁-C₈ alkyl, C₂-C₈ alkenyl, C₂-C₈ alkynyl, C₃-C₈ cycloalkyl, and C₁-C₈ haloalkyl.

19. The method of claim 18, wherein R₄ is CH₂CH═C(CH₃)₂.

20. The method of claim 19, wherein said compound is 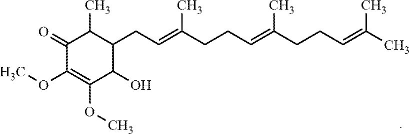
.
